(19) United States
(12) Patent Application Publication
(10) Pub. No.: US 2026/0284609 A1
(43) Pub. Date: Sep. 24, 2026

(54) FLUID PATH SYSTEM, MIXING SYSTEM, METHOD FOR MANUFACTURING A MIXTURE, AND USE OF A FLUID PATH SYSTEM AND OF A MIXING SYSTEM

(71) Applicant: BioNTech SE, Mainz (DE)

(72) Inventors: Heinrich HAAS, Mainz (DE); Sebastian HÖRNER, Mainz (DE); Tobias KIND, Mainz (DE)

(21) Appl. No.: 19/472,352

(22) PCT Filed: Mar. 28, 2024
(86) PCT No.: PCT/EP2024/058504

§ 371 Date: Oct. 3, 2025

(30) Foreign Application Priority Data
Apr. 3, 2023 (WO) PCT/EP2023/058702

(51) Int. Cl. B01F 25/00; B01F 35/71

(52) U.S. Cl. CPC B01F 25/14; B01F 35/712; B01F 35/7174

(57) ABSTRACT

The invention provides a fluid path system, in particular for aseptic processing of at least a first fluid and a second fluid, the fluid path system comprising at least a first tubing unit and a second tubing unit, wherein the first tubing unit is configured to receive the first fluid from a first reservoir and the second tubing unit is configured to receive the second fluid from a second reservoir, a mixing element configured to combine the first fluid and the second fluid to provide a mixture of the first fluid and the second fluid and to discharge the mixture of the first fluid and the second fluid, where filling and emptying the tubes is obtained by modification of the air or gas pressure inside the tubings by gas-filled syringes which are connected to the tubings through sterile filters, such that only sterile air or gas will come into connection with the fluids in the tubings. Furthermore, a mixing system and a corresponding method for mixing of at least two fluids is provided. The invention allows for an aseptic and precise mixing of fluids or fluid phases, in particular of two or more fluids or fluid phases.

### TECHNICAL FIELD OF THE INVENTION

The invention relates to mixing arrangements and techniques for an aseptic mixing of fluids or fluid phases, in particular of two or more fluids or fluid phases. More specifically, the invention relates to a fluid path system, to a mixing system comprising the fluid path system, to a method is for manufacturing a mixture of fluids or fluid phases and to the use of a fluid path system and a mixing system.

### BACKGROUND OF THE INVENTION

Mixing of two or more fluids or fluid phases to manufacture a sterile mixture of these fluids is a common task in the pharmacological or medical field, but also in neighbouring fields such as in general laboratory environments and the like.

For example, mixing processes for mixing for example two fluids are used, where mixing is realized by two or more syringes using separate or coupled syringe pumps. As the syringes used for manufacturing of the mixture are generally not tight with respect to potential bacterial or other contamination, manufacturing has to be performed in an aseptic environment for sterile processing of fluids. Thus, manufacturing is for example performed in an isolator in C-class or D-class environment, or in a sterile laminar flow hood in a B-class environment. However, such set-up does not allow for high-throughput manufacturing of, e.g., several batches per day, as extensive and tedious cleaning of the isolator between two batches and prolonged sterilization procedures in the isolator are required. That is, the known systems and their handling is complex and time-consuming.

Pumping may also be performed with other pumping systems. For example, in pharmaceutical processing, peristaltic pumps are frequently used. In principle, peristaltic pumps allow as well sterile processing of fluids. However, these pumping systems are characterized by pronounced oscillations of the pumping speed and pressure, resulting in unacceptable changes of the mixing conditions. Damping of the oscillations is in principle possible, but it requires technical intervention, which leads to increased complexity, and also the risk of failures increases. Damped peristaltic pumps are therefore less desirable from a control point of view.

### SUMMARY OF THE INVENTION

It is an object of the present invention to provide an arrangement for manufacturing a sterile mixture of two or more fluids or fluid phases, in particular of two or more liquids, wherein the arrangement comprises a simple structure, is easy to handle in any type of environment and allows for achieving precise mixing ratios irrespective of the batch sizes. More specifically, the invention allows manufacturing of very small batch sizes as well as large batch sizes by means of continuous pumping. It is in particular an object of the present invention, that variations of the mixing ratio are avoided and that the mixing ratio is maintained with high temporal resolution, e.g., variations in mixing ratio due to high frequent oscillations are avoided. It is a further object of the present invention to provide a corresponding use of the arrangement and a corresponding method for manufacturing of a sterile mixture of two or more fluids, in particular of two or more liquids.

To achieve these and other objects of the present invention, the following is provided:

In one aspect, the invention provides a fluid path system, in particular for aseptic processing of at least a first fluid and a second fluid. The fluid path system comprises at least a first tubing unit and a second tubing unit, wherein the first tubing unit is configured to receive the first fluid and the second tubing unit is configured to receive the second fluid. The fluid path system further comprises a mixing element configured to combine the first fluid and the second fluid to provide a mixture of the first fluid and the second fluid and to discharge the mixture of the first fluid and the second fluid, and at least one sterile filter unit configured to allow passage of an auxiliary medium or to supply an auxiliary medium, such that a sterile auxiliary medium is supplied or can be supplied to the first tubing unit and to the second tubing unit.

In one further aspect, the invention provides a fluid path system, in particular for aseptic processing of at least a first fluid and a second fluid. The fluid path system comprises at least a first tubing unit and a second tubing unit, wherein the first tubing unit is configured to receive the first fluid and the second tubing unit is configured to receive the second fluid. The fluid path system further comprises a mixing element configured to combine the first fluid and the second fluid to provide a mixture of the first fluid and the second fluid and to discharge the mixture of the first fluid and the second fluid, and at least one sterile filter unit configured to allow passage of a sterile auxiliary medium for transmitting or applying a pressure to the tubing units, such that the first fluid and the second fluid are pumpable through the fluid path system and that the mixture of the first fluid and second fluid is provided.

In one further aspect, the invention provides a fluid path system, in particular for aseptic processing of at least a first fluid and a second fluid. The fluid path system comprises at least a first tubing unit and a second tubing unit, wherein the first tubing unit is configured to receive the first fluid and the second tubing unit is configured to receive the second fluid. The fluid path system further comprises a mixing element configured to combine the first fluid and the second fluid to provide a mixture of the first fluid and the second fluid and to discharge the mixture of the first fluid and the second fluid, and at least one sterile filter unit configured to allow passage of a sterile auxiliary medium or to supply a sterile auxiliary medium for transmitting or applying a pressure to the tubing units, such that the first fluid is receivable in the first tubing unit, the second fluid is receivable in the second tubing unit, the first fluid and the second fluid are combinable in the mixing element and/or the mixture is dischargeable from the mixing element.

In one further aspect, the invention provides a fluid path system, in particular for aseptic processing of at least a first fluid and a second fluid. The fluid path system comprises at least a first tubing unit and a second tubing unit, wherein the first tubing unit is configured to receive the first fluid and the second tubing unit is configured to receive the second fluid. The fluid path system further comprises a mixing element configured to combine the first fluid and the second fluid to provide a mixture of the first fluid and the second fluid and to discharge the mixture of the first fluid and the second fluid, and at least one sterile filter unit configured to allow passage of a sterile auxiliary medium or to supply a sterile auxiliary medium which mediates one or more of the following: filling the first tubing unit with the first fluid, filling the second tubing unit with the second fluid, combining the first fluid and the second fluid in the mixing element, and discharging the mixture of the first fluid and the second fluid from the mixing element.

The inventive arrangement allows for a reliable control of mixing under sterile conditions. Reproducibility of manufacturing and safety of the product is given to a high degree, in particular, if the tubing units are operated in parallel, that is, simultaneously. Thus, the risk of changes in product quality is minimized and a sterile mixture of the first fluid and the second fluid, for example of a first liquid or a second liquid, is obtained.

The fluid path system according to all of these aspects may comprise at least a first tubing unit and a second tubing unit. That is, in various embodiments of all aspects, more than a first fluid and a second fluid, for example a third or fourth fluid, may be mixed with the fluid path systems of the invention. Thus, further tubing units may be provided, such as a third, a fourth, a fifth, a sixth or more tubing units. The further tubing units may also process the first fluid and the second fluid. In a further embodiment of all aspects, the third tubing unit may process a third fluid, the fourth tubing unit may process a fourth fluid etc. In one embodiment of all aspects, the system may also comprise two or more mixing elements.

The tubing units of all aspects may comprise or may be provided as tube elements or tubes for processing the fluids. In one embodiment of all aspects, the first tubing unit is provided as a first tube element or tube and the second tubing unit is provided as a second tube element or tube. In other embodiments of all aspects as described in more detail below, each tubing unit may comprise more than one tube element or tube, for example two, three, four or more tube elements. The tube elements or tubes are preferably air-filled sterile tube elements or tubes.

The fluid path from the first or second fluid reservoir to the mixing element may also be provided as a first or second fluid path, including for example the corresponding tubing units, that is the first tubing unit and the second tubing unit.

It should be noted that also the mixture is a fluid. At least a first fluid and a second fluid may be combined or mixed to produce a further fluid, which is the mixture of the first fluid and the second fluid or any further fluid.

In one embodiment of all aspects of the invention, one of the fluids is a liposome colloid and the other fluid is an RNA solution. optionally a NaCl containing RNA solution. For example, the first fluid is a liposome colloid, and the second fluid is an RNA solution, optionally a NaCl containing solution. Thus, the finished mixture of the first fluid and the second fluid contains lipoplex particles (LPX), for example LPX in RNA and liposome buffer.

That is, in one embodiment of the disclosure, a fluid path system is used for GMP- (good manufacturing practice) manufacturing of the pharmaceutical RNA lipoplex particle product, which enables accurate control of the mixing ratio of RNA to liposomes, which is important for product quality. In one embodiment, mixing of a liposome solution and an RNA solution in a 1 to 1 (volume/volume) manner is provided, where the concentrations of the components are selected in order to exactly maintain the intended charge ratio.

The auxiliary medium may be provided for transmitting or applying a pressure to the fluid path system, in particular to the tubing units, in particular to the fluids, for example to the liquids. The auxiliary medium allows for transporting or conveying the fluids through the fluid path system. Thus, the fluids can be conveyed or transported within the corresponding fluid path system so that the fluids can be combined or mixed and so that a mixture of the at least first fluid and second fluid can be discharged.

In one embodiment of all aspects of the invention, the auxiliary medium comprises a gas, e.g. air or a gas different than air, or is air. Gas or air is easy to handle and can be filtered in a desired manner by the at least one filter unit. In one embodiment of all aspects, the auxiliary medium is a non-sterile medium and comprises any kind of fluidic medium, including gaseous or liquid medium, and mixtures thereof. In one embodiment of all aspects, the auxiliary medium comprises a mixture of air and/or one or more other gaseous media such as industrial gas. The industrial gas may, for example, comprise nitrogen, oxygen, carbon dioxide, argon, hydrogen, helium, and mixtures thereof, as may, for example, be provided for medical use.

The mixing element of all aspects may be configured or is configured to mix the at least first and second fluids, for example by combining the fluids to be mixed, in particular by combining or mixing a first liquid and a second liquid or any further liquid. The wordings “combining fluids in the mixing element” or “the combination of the fluids in the mixing element” as used herein means that a mixture of the fluids to be mixed, for example of the first fluid and the second fluid, is provided. In the simplest form, combining of fluids means mixing the fluids by bringing the fluids together.

In one embodiment of all aspects of the invention, at least the first tubing unit, the second tubing unit and the mixing element are provided as a tubing set. In one embodiment of all aspects, the tubing set is pre-sterilized. In one embodiment of all aspects the fluid path system and/or the tubing set is/are pre-assembled, including in particular bags, connector elements, valve elements, etc.

It should be noted that in particular in pharmaceutical applications, the fluid path system has to be configured to allow for an aseptic manufacturing of the mixture of the at least first and second fluids. Thus, the fluid path system or the sterile fluid path system according to all of these aspects may comprise sterile fluid paths. That is, in one embodiment of all aspects, the fluid path system is a sterile fluid path system or is a pre-sterilized fluid path system. The invention, thus, allows for an aseptic and precise mixing of fluids or fluid phases, in particular of two or more fluids or fluid phases.

In various embodiments of all aspects of the invention, the equipment, that is, in particular the components of the fluid path system, are pre-sterilized (tubing units, mixing elements, bags etc.), and are preferably pre-assembled, and are preferably available from established manufacturers.

In one embodiment of all aspects, the sterile filter unit is configured to supply a sterile auxiliary medium to the first tubing unit and to the second tubing unit. Aseptic mixing in the fluid path system of all of the above aspects is, amongst others, achieved by the one or more sterile filter units, since only a sterile auxiliary medium is conveyed or supplied to the tubing units, or, in other words, since any medium which enters the fluid path system for conveying the fluids within the fluid path system is filtered by passing the sterile filter unit.

The sterile filter unit of all aspects may be configured or is configured to allow the passage of the auxiliary medium, such that the output of the sterile filter unit is a sterile auxiliary medium. The auxiliary medium when in contact with the fluids, i.e. the first fluid and/or the second fluid, may therefore be sterile, and therefore any risk of bacterial contamination of the sterile fluids, for example the liquids, in the tube elements or the fluid paths is excluded. This allows for obtaining a sterile mixture of the first fluid and the second fluid.

In one embodiment of all aspects, the at least one sterile filter unit is configured to prevent, that is, to block, passage of the first fluid and/or the second fluid. In other words, the sterile filter unit does not allow passage of the first fluid and/or second fluid. None of the fluids may leak out via the sterile filter unit. Merely the auxiliary medium is conveyed, for example from a pumping unit, via the at least one sterile filter unit to the tubing units or from the tubing units via the at least one sterile filter unit to the pumping unit.

In one embodiment of all aspects of the invention, the at least one sterile filter unit is configured to allow the passage of the auxiliary medium for transmitting or applying a pressure to the fluid path system, in particular to the tubing units, in particular to the at least first fluid and second fluid, such that the first fluid is receivable in the first tubing unit, the second fluid is receivable in the second tubing unit, the first fluid and the second fluid are combinable or mixable in the mixing element and/or that the mixture of the first fluid and the second fluid is discharged or can be discharged from the mixing element. In one embodiment of all aspects, the auxiliary medium is conveyed to the tubing units via a pumping unit. The pumping unit allows for controlling of the mixing process.

In one embodiment of all aspects, the sterile filter unit is an air or gas filter.

In one embodiment of all aspects, the at least one sterile filter unit comprises one or more sterile filter elements, in particular at least a first sterile filter element and a second sterile filter element,
wherein, optionally, the at least one sterile filter unit comprises
i) a first sterile filter element or a first sterile filter associated with the first tubing unit and a second sterile filter element or a second sterile filter associated with the second tubing unit, or
ii) a sterile filter element or a sterile filter associated with the first tubing unit and the second tubing unit.

In one embodiment of all aspects, the fluid path system is configured to be connected or is connectable to a pumping unit, so that the auxiliary medium is suppliable or can be supplied to or is removable from the tubing units.

In one embodiment of all aspects, the fluid path system is configured to be connected or is connectable to the or a pumping unit, so that the fluids to be mixed can be pumped through the fluid path system.

In one embodiment of all aspects of the invention, the fluid path system is configured to be connected or is connectable to the or a pumping unit or system, as already explained above. In particular, the first tubing unit and the second tubing unit are connectable to the pumping unit via the sterile filter unit. The pumping unit is configured to supply the auxiliary medium to or remove the auxiliary medium from the fluid path system, and thus, to or from the tubing set, thereby allowing the fluids to be mixed to be conveyed through the fluid path system. Supplying and/or removing the auxiliary medium to or from the tubing units and thus, to or from the tubing set of the fluid path system, allows for pumping, that is for conveying the fluids by transmitting a pressure to the fluids. This causes movement of the fluids such that the first fluid and the second fluid can be combined or can be mixed and that the mixture of the first fluid and the second fluid is discharged or can be discharged from the mixing element. The pumping unit is preferably configured to control the auxiliary medium, for example air or a gas, that is to control supply and removal of the auxiliary medium to the tubing units.

In other words, the passage of a sterile auxiliary medium through the sterile filter may increase or decrease the pressure in the tubing units with respect to e.g. the environmental pressure. By decreasing the pressure with respect to environmental pressure, the first fluid can be received in the first tubing unit from a reservoir exposed to environmental pressure, such as a flexible bag, and the second fluid can be received in the second tubing unit from a reservoir exposed to environmental pressure, such as a flexible bag. By increasing the pressure, the first fluid and the second fluid can be combined in the mixing element and/or the mixture can be discharged from the mixing element to a reservoir exposed to environmental pressure, such as a flexible bag.

The pressure, preferably air or gas pressure, at certain parts of the closed sterile fluid path system can thus be either increased or decreased, thus allowing to process the fluids into or inside the sterile fluid path system. That is, the external pumping unit which can be arranged in a non-sterile environment provides a pressure gradient, in particular an air or gas pressure gradient. Processing and/or pumping is preferably performed in all aspects by a pneumatic pressure gradient, which is applied by air or gas cushions. That is, the fluids are preferably pneumatically pumped using sterile air or gas.

Each tubing unit is preferably associated, preferably connected or connectable with a single sterile filter element. As an alternative, only one sterile filter element is provided which is configured to be connected or connectable with each of the tubing units. In use, the at least one filter unit is arranged between the pumping unit and the tubing units, in order to avoid contamination of the fluid path system by the auxiliary medium.

In one embodiment of all aspects of the invention, the pumping unit comprises two or more syringe elements, for example, at least a first syringe element and a second syringe element, wherein, optionally, the first tubing unit is connectable to the first sterile filter element and the first sterile filter element is connectable to the first syringe element, and the second tubing unit is connectable to the second sterile filter element and the second sterile filter element is connectable to the second syringe element. Thus, each tubing unit can be operated by means of a syringe or a syringe element, wherein the operation of the tubing units is preferably carried out in parallel.

Also another combination of the elements is possible, thus, one of the tubing units is connectable or is connected to one of the sterile filter elements and this sterile filter element is connectable or is connected to one of the syringe elements. Furthermore, another, that is, a different tubing unit is connectable or is connected to a different sterile filter element, and this sterile filter element is connectable or is connected to a different syringe element. In any way, a separate fluid path from the reservoir to the mixing element has to be provided for each fluid.

The syringe elements are not in contact with any other medium except the auxiliary medium. For example, the syringe elements may not be in contact with any fluid or mixture of the fluids processed by or through the fluid path system. The syringe elements allow for an effective pumping process, in particular, shear forces can be avoided.

The pumping unit which is used with the sterile fluid path system according to all aspects as described above is preferably configured to drive the syringe elements in parallel. A parallel operation of the syringe elements, that is, pumping at the same time, allows for applying an identical or substantially identical pressure to the fluids in the tubing units and thus for a precise and efficient mixing process. The tubing units or tube elements are thus preferably provided as identical tubing units.

In one embodiment of all aspects of the invention, the fluid path system further comprises two or more fluid reservoirs, in particular a first fluid reservoir and a second fluid reservoir, wherein, optionally, the first fluid reservoir containing the first fluid is connectable to the first tubing unit and the second fluid reservoir containing the second fluid is connectable to the second tubing unit. In case that more than two fluids or fluid phases should be mixed, further reservoirs can be provided.

The reservoirs which contain the fluids to be mixed can be part of the fluid path system or can be provided as separate components which are connectable to the fluid path system.

In a further embodiment of all aspects, the fluid path system further comprises at least one container, collection bag or product bag for the mixture of the first fluid and the second fluid, that is, for receiving the mixture, that is, the product manufactured from the first and second fluids and/or the further fluids. The at least one container is connectable to the mixing element. The at least one container may also be part of the fluid path system or can be provided as separate component which is connectable to the fluid path system. The container or bag may be disconnected from the tubing set in aseptic manner in order to maintain sterility inside the container or bag.

Reservoirs, containers and/or bags and/or the fluid path system are configured so that the reservoirs, containers and/or bags can be connected or disconnected to the fluid path system in an aseptic manner, in order to maintain sterility inside reservoirs, containers and/or bags.

In one embodiment of all aspects of the invention, the fluid path system further comprises one or more sterile connector elements for sterile connecting the elements of the fluid path system. In particular, the connector elements allow for connecting the first fluid reservoir for the first fluid to the first tubing unit, the second fluid reservoir for the second fluid to the second tubing unit or, if necessary, vice versa, and/or the at least one container for the mixture of the first fluid and the second fluid to the mixing element. Thus, contamination can be avoided when the reservoirs and/or the container(s) are not part of the fluid path system and when these components are connected to the fluid path system. Sterile connector elements may also be provided for connecting the sterile filter unit to the pumping unit.

In one embodiment of all aspects of the invention, the fluid path system comprises one or more connecting tubes which are configured to connect the components or elements of the fluid path system to each other or to external components or elements. Connecting tubes may be arranged at the reservoirs or container, at the tubing units and/or at the mixing element.

In one embodiment of all aspects of the invention, said reservoirs comprise bags, bottles, and the like, and the sterile connector elements are adapted for sterile connection of said bags or bottles to the tubing set of the fluid path system.

In one embodiment of all aspects of the invention, the fluid path system further comprises one or more valve elements, wherein at least one valve element is arranged
i) between each tubing unit and an associated fluid reservoir, and/or
ii) between each tubing unit and the mixing element, and/or
iii) between the mixing element and the container for the mixture of the first fluid and the second fluid.

The valve elements are preferably provided as external elements, in particular outside of the fluid path. The valve elements are preferably arranged such that the fluids can be pumped into different directions.

In one embodiment of all aspects, the fluid path system further comprises one or more valve elements for allowing and/or preventing one or more of the following: the input of the first fluid, the input of the second fluid, the combination of the first fluid and the second fluid and the output of the mixture of the first fluid and the second fluid.

In one embodiment of all aspects of the invention, the fluid path system further comprises one or more valve elements for allowing and/or preventing one or more of the following: filling the first tubing unit with the first fluid, filling the second tubing unit with the second fluid, combining the first fluid and the second fluid in the mixing element and discharging the mixture of the first fluid and the second fluid from the mixing element.

In one embodiment of all aspects, the fluids from the reservoirs to be mixed may be, in a first step, filled into the corresponding tubing units and, in a second step, may be discharged from the tubing units and conveyed to the mixing element, and, in a third step, may be combined in the mixing element and discharged from the mixing element to provide the mixture of the fluids to be mixed. Since the fluids are conveyed in different directions during the mixing process, it is necessary to open or to close corresponding fluid paths within the fluid path system in order to convey the fluids in the desired direction.

In case of a first and a second fluid, the following mixing procedure may be carried out with the inventive arrangement, that is, with a sterile fluid path system according to any one of the above aspects and embodiments. A fluid path system of each aspect is suitable for carrying out the following process:
a) opening a first valve element between the first tubing unit and the associated first fluid reservoir, and opening a second valve element between the second tubing unit and the associated second fluid reservoir,
b) closing a third valve element between the first tubing unit and the mixing element, and closing a fourth valve element between the second tubing unit and the mixing element,
c) pumping the first fluid into the first tubing unit, and pumping the second fluid into the second tubing unit,
d) closing the first valve element and the second valve element,
e) opening the third valve element and the fourth valve element,
f) pumping the first fluid from the first tubing unit through the mixing element, and pumping the second fluid from the second tubing unit through the mixing element, preferably in parallel,
g) combining the first fluid and the second fluid in the mixing element to provide a mixture of the first fluid and the second fluid, and discharging the mixture of the first fluid and the second fluid from the mixing element.

Opening of a valve element allows the fluid and the mixture, which is also a fluid, to flow through the fluid path; closing of a valve element blocks the flow of fluid through the fluid path.

For operating the sterile fluid path system, the sterile fluid path system is connected to a specific mechanism which allows for conveying the first and second fluids, that is, for example to a pumping unit. The pumping unit preferably comprises air or gas filled syringe elements or air or gas-filled syringe pumps, which are coupled or connected to the fluid path system by the sterile filter unit, in particular by the sterile filter elements. In case of a first and a second fluid, the following mixing procedure may be carried out with the inventive arrangement, that is, with a sterile fluid path system according to any one of the above aspects:
a) initially, the syringe elements are emptied,
b) first reservoir containing the first fluid and second reservoir, containing the second fluid, for example liposome and RNA bags, are connected by sterile connector elements,
c) valve elements are provided to allow filling and emptying the tubing units,
d) valve elements are adjusted as described above under items a) and b),
e) then syringe elements are pulled open, thus fluids (liposomes, RNA) are sucked into the tubing units of the fluid path system; tubing units are preferably long enough to hold the complete fluid to be manufactured,
f) valve elements are switched as described above under items d) and e), that is connection to bags are closed, and opened into the direction of the mixing element,
g) the two (air- or gas-filled) syringe elements are emptied, in particular in parallel; the air or gas passes through the sterile filters and applies pressure on the fluids, in particular liquids, in the tubing units, which pushes them through the mixing element, for example a V-mixer, in particular in parallel.

Filling and emptying the tubing units with the corresponding fluid may be performed by air- or gas-filled syringes, which are connected through sterile filters to the fluid path system through the sterile filters. Increasing or decreasing of the volume inside the syringes may lead to increase or decrease of pressure of the air or gas cushion in contact with the fluids in the tubing, thus applying a pressure gradient to the tubing units and thus to the fluids, in particular by decreasing or increasing the pressure inside the tubing units for filling the tubing units with the corresponding fluid and by increasing the pressure inside the tubing units for emptying the tubing units. Thus, the fluids to be mixed are driven by pressure, in particular air or gas pressure.

This process ensures mixing, that is combining, of the two fluids with accurately controlled absolute and relative flux.

In one embodiment of all aspects, the one or more valve elements comprise one or more of the following: a three-way valve, a T-piece, a clamp, and a pinch valve. That is, three-way valves, T-pieces, clamps, pinch valves or also stopcocks and the like can be used for allowing and/or preventing the flow of the fluids.

According to various embodiments of all aspects of the invention, valve elements and/or the connector elements are configured to permit filling the tubing set with fluids from bags, bottles, etc. According to embodiments, this may additionally allow adding one or more further ingredients such as, for example, a cryoprotectant or a buffer to the mixture of the first and second or more fluids.

In one embodiment of all aspects of the invention, the mixing element comprises a manifold valve or a mixing tee, a Y-mixer or a T-mixer.

In one embodiment of all aspects of the invention, one or more of the following are single use elements, that is disposable elements: the first tubing unit, the second tubing unit or any further tubing unit, or at least parts of the tubing units, the mixing element, the at least one sterile filter unit, the one or more valve elements and the one or more connector elements. Depending on the set-up of the fluid path system, one or more components can be replaced after use, in order to allow for an aseptic mixing procedure. Preferably, all of the elements of the fluid path system are single use elements. The required equipment, in particular single use elements, are available at low costs. That is, in one embodiment, a mixing setup is realized, which is fully based on single use materials.

Thus, mixing is possible under aseptic conditions, using for example the single use equipment. The inventive set-up can be established also in an environment which does not fulfil the criteria for aseptic processing. More specifically, manufacturing of the mixture does not require so-called A-class environment, the mixing procedure is also possible in B-class or even C-class or D-class environment.

In one embodiment of all aspects of the invention, one or more of the following are sterile or pre-sterilized elements: the first tubing unit, the second tubing unit or any further tubing unit, the mixing element, the at least one sterile filter unit, the one or more valve elements and the one or more connector elements. Preferably, all of the elements of the fluid path system are sterile elements. Thus, a pharma grade system is provided.

According to embodiments of all aspects, the single use material is material available from established providers in pre-sterilized form, in particular, the components are GMP (good manufacturing practice) compliant components. According to embodiments of all aspects, some or all of the elements included in the inventive fluid path system or setup may be pre-sterilized. According to embodiments of all aspects, some or all of the elements included in the inventive fluid path system or setup may be pre-assembled.

In one embodiment of all aspects of the invention, one or more of the elements of the fluid path system are not connected to any other element or component. In a further embodiment of all aspects, one or more of the elements or components are packaged individually. In a further embodiment of all aspects, one or more of the elements or components are packaged together as a set. In a further embodiment of all aspects, all elements or components are packaged as a set.

In one embodiment of all aspects of the invention, the first tubing unit is configured for complete input of the first fluid and/or the second tubing unit is configured for complete input of the second fluid. In one embodiment of all aspects of the invention, the first tubing unit and/or the second tubing unit comprise(s) a storage capacity such that a predetermined amount of a respective fluid is storable within the first tubing unit and/or the second tubing unit. The tubing units may be, configured to receive the complete content of one or more of the associated reservoirs of fluid to be mixed. In this way, the mixture of the first and second fluids or any further fluid can be obtained with one mixing cycle. Thus, accuracy of the mixing ratio can be increased.

In one embodiment of all aspects, the first tubing unit may be configured to receive the complete first fluid, for example according to a given batch size and/or mixing ratio, and the second tubing unit may be configured to receive the complete second fluid, for example according to a given batch size and/or mixing ratio. That is, each of the tubing units may be configured to accommodate the volume of the corresponding fluid intended for mixing.

According to various embodiments of all aspects, the volume, e.g. length and/or inner diameter, of the first tubing unit and the second tubing unit or any further tubing unit between the mixer and the sterile filter unit is dimensioned, such that the tubing units can hold the whole volume of the syringes coupled to the tubing parts in direct contact with the syringes, e.g., using 100 mL syringes two phases of 100 ml, can be mixed by one full stroke of the syringes.

According to various embodiments of all aspects, the first tubing unit and the second tubing unit or any further tubing unit may comprise a storage capacity which is not dimensioned to receive the complete fluid or fluids to be processed. Thus, more than one pumping cycle, for example a repeated activation, e.g. a pulling and pushing of pistons of the syringe elements, is required to manufacture the required batch size. For example, if the tubing set is configured to receive one half, one third, etc. of the desired batch, two times, three times, etc. pulling and pushing would be required. Any other fractional configuration is possible.

In one embodiment of all aspects of the invention, each tubing unit may comprise or may be provided as one or more tube elements, for example one or more tubes. For example, each tubing unit may comprise one tube element, two tube elements, three tube elements, four tube elements etc. In one embodiment of all aspects, each tubing unit comprises or is provided as two tube elements which are both connectable to one or to a respective fluid reservoir, and wherein each tube element is connectable to a pumping unit, in particular to a syringe element or a syringe pump, wherein each tube element is connectable to a different syringe element or a syringe pump. In order to allow for an aseptic mixing process, the tube elements are connected to the pumping unit via the sterile filter unit.

The first tubing unit may, in one embodiment of all aspects, comprise two tube elements and the second tubing unit may also comprise two tube elements for processing the corresponding fluids. The first fluid may be for example conveyed via a first and a second tube element of the first tubing unit and the second fluid may be for example conveyed via a first and a second tube element of the second tubing unit. The two or more tube elements or tubes of the first tubing to unit and the two or more tube elements or tubes of the second tubing unit allow for a continuous flow of the first and second fluids within the fluid path system and for continuous discharging of the mixture of the first and second fluids. Preferably, an uninterruptible conveying of the fluids is provided until the desired amount of the mixture of the first fluid and the second fluid, or of a further fluid, is obtained.

In case of, for example, two tube elements for each tubing unit, pumping is controlled such that fluid is conveyed to the mixing element at any time in a continuous flow. In one embodiment of all aspects, each tube element of each tubing unit is for example connected to a corresponding syringe element of the pumping unit via a corresponding sterile filter element. The syringe elements of each tube element of each tubing unit are preferably activated in such a manner that the bottom dead center of one of the syringe elements and the top dead center of the other of the syringe elements are reached at different times. Thus, a continuous flow is guaranteed without any interruption. The top dead center may be the point at which the plunger of a syringe is nearest to the outlet of the syringe. The bottom dead center may be the point at which the plunger of a syringe is at it furthest form the outlet of the syringe.

In one embodiment of all aspects of the invention, the fluid path system further comprises a sterile enclosure or sterile bag, wherein at least the first tubing unit, the second tubing unit, any further tubing unit, if any, and/or the mixing element or the one or more mixing elements are arranged within the sterile enclosure or sterile bag. In this case, the sterile enclosure or sterile bag is accessible from the outside, that is, the tubing units are connectable to the pumping unit via the one or more sterile filter elements. Furthermore, the fluid reservoirs are also connectable to the tubing units, preferably via sterile connector elements.

In one further embodiment of all aspects the two or more fluid reservoirs and/or the at least one container for receiving the mixture of the fluids is/are arranged within the sterile enclosure of sterile bag. The arrangement may be in particular provided as a disposable fluid path system and may allow for the manufacturing of a mixture of the first and second fluids by means of for example one mixing cycle.

In one embodiment of all aspects of the invention, the fluid path system of the invention comprises pharma grade soft tubes and/or pharma grade bags.

In one embodiment of all aspects of the invention, the ratio of the volumes of the two fluids is 1 to 1 with the lowest possible variation, preferably <5%, more preferably <2%. Thus, it is possible to maintain the molar ratio of the components, dissolved in the two phases, at a fixed value with the lowest possible variation. Even variations for very short time, for example milliseconds, can be avoided with the inventive arrangement.

In one embodiment of all aspects of the invention, the at least first tubing unit and second tubing unit or any further tubing unit comprise the same shape, size, volume and/or storage capacity. In particular, the tubing units within a fluid path system are identical; thus, a precise mixing process can be carried out.

The fluid path system as described above may be used for aseptic manufacturing of a sterile mixture of a first fluid and a second fluid, or of at least a first fluid and a second fluid. Thus, further fluids may be processed with the inventive arrangement.

With the inventive arrangement according to all aspects as described herein, the following can be realized:
only one pumping stroke can be used for mixing a desired batch size, and/or
the fluids may be pumped or conveyed through the fluid path system in parallel; that is, the tubing units can be operated in parallel or simultaneously, and/or
a sterile mixture, e.g. the product, can be manufactured.

In one aspect, the invention provides a mixing system comprising the fluid path system of the invention, that is, a fluid path system according to any of the aspects as described above, and a pumping unit for pumping a first fluid and a second fluid through the fluid path system. The pumping unit is configured to or allows for pumping the first fluid, the second fluid or further fluid(s) through the fluid path system, such that the first fluid and the second fluid are combined or are combinable and that a mixture of the first fluid and the second fluid is discharged or can be discharged from the mixing element.

The invention refers preferably to the manufacturing of for example lipoplex nanoparticles from two or more fluid phases using the mixing system and the fluid path system.

In one embodiment, the pumping unit is configured to provide a pressure or a pressure gradient, in particular a pneumatic pressure gradient, in particular via the auxiliary medium, which is preferably air or gas, to the fluid path system, in particular to the tubing units, in particular to the fluids to be mixed. The air or gas pressure at certain parts of the closed sterile fluid path system, in particular achieved via air or gas cushions, can be either increased or decreased, thus allowing to process the fluids into or inside the sterile fluid path system. Since the sterile filter unit is provided, the external pumping unit can be arranged in a non-sterile environment.

In one embodiment of the invention, the pumping unit comprises two or more syringe elements, in particular at least a first syringe element and a second syringe element, wherein, optionally, the first tubing unit is connectable to the first sterile filter element and the first sterile filter element is connectable to the first syringe element, and the second tubing unit is connectable to the second sterile filter element and the second sterile filter element is connectable to the second syringe element.

The syringe elements may be used for pumping. The syringe elements may not be in contact with any other medium except the auxiliary medium. For example, the syringe elements may not be in contact with any fluid or mixture of the fluids processed by or through the fluid path system.

The pumping unit, for example a double syringe pump actuator, may be configured to supply an auxiliary medium, preferably air or gas, to the first tubing unit and to the second tubing unit and/or to remove the auxiliary medium from the first tubing unit and to the second tubing unit.

Supplying and/or removing the auxiliary medium to or from the tubing units and thus, to or from the tubing set of the fluid path system, allows for pumping, that is for conveying the fluids by transmitting a pressure to the fluids. This causes movement of the fluids such that the first fluid and the second fluid are mixed or can be mixed or combined and that the mixture of the first fluid and the second fluid is discharged or can be discharged from the mixing element. The pumping unit is preferably configured to control the auxiliary medium, for example air or gas, that is to control supply and removal of the auxiliary medium to the tube elements.

The pumping unit is preferably configured to introduce the fluids, for example liquids, into the sterile fluid path system by lowering or decreasing the pressure inside certain parts of the sterile fluid path system, in particular inside the tubing units. The fluids inside the sterile fluid path system can be processed and mixed by elevating or increasing the pressure inside the certain parts of the sterile fluid path system, in particular inside the tubing units.

The pumping unit is preferably configured to drive its syringe elements in parallel. A parallel operation of the syringe elements allows for applying an identical or substantially identical pressure to the fluids in the tubing units and thus for a precise and efficient mixing process. The tubing units are thus preferably provided as identical tubing units.

In one embodiment of all aspects, each tubing unit comprises at least two tube elements which are both connectable to a respective fluid reservoir or to different fluid reservoirs, and wherein each tube element is connectable to a syringe element, wherein each tube element is connectable to a different syringe element. Each tube element is connectable to a syringe element, as described above via the sterile filter unit. With such an arrangement and a corresponding control of the pumping procedure, a continuous flow of the fluids without any interruption can be provided. That is, two syringe elements or pumps are provided for each feed. The syringe elements of each tube element of each tubing unit are preferably activated in such a manner that the bottom dead center of one of the syringe elements and the top dead center of the other of the syringe elements are reached at different times. In particular, the syringe elements of each tube element of each tubing unit are preferably activated in such a manner that a continuous flow of the fluids to be mixed is provided, that is, without any interruption.

In one embodiment of all aspects of the invention, the mixing system further comprises
(i) a second pumping unit, optionally a peristaltic pump, for supplying a substance to the at least one container, and/or
(ii) a rocker device, wherein the at least one container is receivable in the rocker device, and/or
(iii) a third pumping unit, optionally a peristaltic pump, for pumping the mixture from the container to a further container, preferably via a further filter element.

In this case, the container may be a first container or collection or product bag, that is, the container for receiving the mixture of the at least first fluid and second fluid. The further container may be a second container or product bag for receiving a ready to use mixture. It should be noted that also the mixture without any additional substance, that is the mixture in the first container may be a ready to use mixture.

In one embodiment of all aspects, the pumping unit is configured to drive the syringe elements in parallel.

In one embodiment of all aspects, the mixing system and/or the fluid path system is configured to manufacture or to provide the mixture of the fluids to be mixed by means of one pumping cycle, that is, by using one pumping cycle. In particular, the mixing system allows for manufacturing a predefined batch size of the mixture with one pull-/push-operation of the syringe elements.

In one embodiment of all aspects, the fluid path system and/or the mixing system may comprise instructions for use of the fluid path system and/or the mixing system, preferably including how to connect one or more of the connectable elements with each other and/or elements not included in the fluid path system and/or the mixing system.

In one embodiment of all aspects, one of the fluids is a liposome colloid and the other fluid is an RNA solution, optionally a NaCl containing solution.

In one embodiment, the at least first tubing unit and second tubing unit or any further tubing unit comprise the same shape, size, volume and/or storage capacity. In particular, the tubing units within a fluid path system are identical; thus, a precise mixing process can be carried out.

In one embodiment of all aspects of the invention, and as already explained above, the at least one sterile filter unit is for example connected or connectable in between the pumping unit and the tubing set, in particular in between the pumping unit and the tubing units. In this way, the pumping unit is decoupled from the sterile fluid path. In one embodiment of all aspects of the invention, the sterile filter unit is arranged at an end of the first tubing unit and/or at an end of the second tubing unit, adjacent to the pumping unit. In other words, the sterile filter unit is arrangeable between the pumping unit and the tubing set, in particular between the pumping unit and the tubing units.

In one embodiment of all aspects of the invention, the at least first and second sterile filter elements of a sterile filter unit are arrangeable so that a respective sterile filter element is arranged between a syringe element of the pumping unit and a respective tubing unit or tube element.

The mixing system as described above may be used for aseptic manufacturing of a sterile mixture of a first fluid and a second fluid, or of at least a first fluid and a second fluid. Thus, further fluids may be processed with the inventive arrangement, for example a third or a fourth or more fluids. That is, all of these fluids can be mixed in the system.

With the inventive systems, the fluid path system and the mixing system, it is possible that the fluids, for example the feed liquids, are supplied via sterile connector elements to a pre-sterilized fluid path system, and thus, to a sterile fluid path system, and that the fluids are aseptically processed.

The inventive arrangement and techniques, that is the mixing system and the fluid path system according to all of the described aspects, allow for an aseptic manufacturing of a mixture of fluids. The process is intrinsically pulsation-free, in particular, if the pumping unit is operated such that the fluids to be mixed are conveyed through the fluid path system in parallel and in particular if mixing is carried out with one mixing cycle, that is, with one piston stroke of the syringe elements. No shear forces occur, as typically occur with peristaltic pumps and the mixing ratio between the phases can be controlled with utmost accuracy, with, for example, variations <2%. Oscillations and deviations from a desired mixing ratio, even for very short time periods, for example, milliseconds (ms), can be avoided. Manufacturing also of small batch sizes is possible, for example, a mixing of volumes of 100 ml or lower or of 50 ml or lower. No complex cleaning procedures or preparation procedures are required. The arrangement and techniques according to the invention can be operated in an economic manner, as no complex equipment, in particular no complex single use equipment, is required. Furthermore, the pumping and/or mixing conditions can be controlled, in particular precisely controlled, at any time, in particular in case of applying pressure to the tubing units simultaneously, that is in parallel. This also allows that the mixing process or procedures are intrinsically safe.

Thus, with the inventive systems, the mixing system and the fluid path system, and with the inventive process, one, more of (e.g. an arbitrarily selected plurality of) or all of the following criteria may be fulfilled,
in particular, if only one pumping stroke is used for mixing the desired batch size,
in particular, if the fluids are pumped or conveyed through the fluid path system in parallel; that is, the tubing units are operated in parallel or simultaneously, and/or
if the sterile filter unit is used:
   mixing of at least two fluids, in particular liquids,
   aseptic manufacturing with single use material,
   small batch sizes (<200 ml, <<200 ml), e.g. mixing of two volumes of 100 ml or lower or 50 ml or lower,
   high throughput, manufacturing of several batches (>10) per day, no complex cleaning or preparations required,
   exact control of mixing ratio (<2% variation),
   exact control of pumping and/or mixing conditions,
   highest possible control of pumping and/or mixing conditions,
   deviations are avoided, even for very short time periods (milliseconds),
   no oscillations,
   fluid dynamics are optimized to maintain particle characteristics and to avoid clogging,
   pumping unit completely decoupled from the fluids and mixture by using a sterile filter unit, that is, for example syringe elements are decoupled form the sterile fluid path,
   pumping unit, in particular syringe elements can be non-sterile,
   GMP (good manufacturing practice) manufacturing in B- or C- or D-class cleanrooms possible, that is, manufacturing can be performed according to GMP guidelines, also in B- or C- or D-class cleanrooms,
   reproducibility of manufacturing and safety of product,
   high product quality, and/or
   economic: costs of in particular single use material relatively low.

In one aspect, the invention relates to a use of the fluid path system of the invention for aseptic manufacturing of a sterile mixture of at least a first fluid and a second fluid.

In one aspect, the invention relates to a use of at least one sterile filter unit as part of the fluid path system of the invention for aseptic manufacturing of a sterile mixture of at least a first fluid and a second fluid.

In one aspect, the invention relates to a use of a mixing system of the invention for aseptic manufacturing of a sterile mixture of at least a first fluid and a second fluid.

In one aspect, the invention relates to a method for aseptic manufacturing of a sterile mixture of at least a first fluid and a second fluid, the method comprising the steps of:
filling at least a first tubing unit with the first fluid and a second tubing unit with the second fluid by removing an auxiliary medium, in particular in parallel, from the tubing units,
mixing, in particular combining the first fluid and the second fluid in a mixing element and discharging a mixture of the first fluid and the second fluid from the mixing element by supplying the auxiliary medium, in particular in parallel, via at least one sterile filter unit, preferably via a first sterile filter element associated with the first tubing unit and a second sterile filter element associated with the second tubing unit, to the tubing units.

In one aspect, the invention relates to a method for aseptic manufacturing of a sterile mixture of at least a first fluid and a second fluid by using the fluid path system according to the invention or the mixing system according to the invention, that is, according to any one of the aspects as described herein, the method comprising the steps of:
filling the at least first tubing unit with the first fluid and the second tubing unit with the second fluid by removing the auxiliary medium, in particular in parallel, from the tubing units,
combining the first fluid and the second fluid in the mixing element and discharging a mixture of the first fluid and the second fluid from the mixing element by supplying the auxiliary medium, in particular in parallel, via the at least one sterile filter unit, preferably via the first sterile filter element associated with the first tubing unit and the second sterile filter element associated with the second tubing unit, to the tubing units.

Thus, the first fluid and the second fluid can be combined or mixed, and the mixture of the first fluid and the second fluid is discharged or can be discharged from the mixing element.

The one or more elements of the fluid path system may be connected with each other to provide the fluid path set-up or system for aseptic manufacturing of the mixture of the at least first fluid and second fluid.

In one embodiment of all these aspects, the method further comprises the step of connecting the first tubing unit and the second tubing unit to the or a pumping unit for pumping the first fluid and the second fluid. The pumping unit allows for supplying or removing the auxiliary medium, preferably air or gas, to the tubing units and thus for carrying out the mixing process. In particular, the sterile filter unit has to be positioned between the pumping unit and the tubing units in order to allow for an aseptic mixing process.

In one embodiment of all the aspects, the pumping unit comprises at least a or the first syringe element and a or the second syringe element as pumping units or pumps, preferably one syringe element for each tube element. That is, the method according to the aspects preferably comprise the following further steps: connecting the first tubing unit to the first sterile filter element and the first sterile filter element to the first syringe element, and connecting the second tubing unit to the second sterile filter element and the second sterile filter element to the second syringe element.

It should be noted that according to all aspects as described herein, the connections may also be provided vice versa, that is a first element may also be connected or may also be connectable to a second element, or a second element may also be connected or connectable to a first element. Also a first fluid may be operated in a second element or a second fluid may be operated in a first element. It only has to be considered that a separate fluid path from a corresponding fluid reservoir to the mixing element has to be provided for each fluid.

Connecting the fluid path system to the pumping unit allows for pulling the auxiliary medium, preferably air or gas, out or sucking the auxiliary medium off the system, that is, removing the medium from the first and second tubing units, at least in part, and for pushing the auxiliary medium into the system, that is, into the first and second tubing units. Pulling and pushing is preferably carried out in parallel with respect to the first and second tubing units, so that an accurate mixing process is provided. The sterile filter unit is configured to supply a sterile auxiliary medium to the tubing units. A parallel operation of the syringe elements is for example carried out by a controller. The controller may be part of the pumping unit or may be provided as a separate device.

In one embodiment of all aspects, the syringe elements are for example air- or gas-cushioned to syringe elements. In one embodiment of all aspects, the sterile fluid path system is coupled to the pumping unit via the at least one sterile filter unit. In one embodiment of all aspects, in use, only the auxiliary medium, for example, air or gas, passes through the at least one sterile filter unit. In one embodiment of all aspects, the sterile filter unit comprises at least two sterile filter elements. Preferably one sterile filter element is provided for each tube element.

In one embodiment of all aspects, the method may be implemented based on the pumping unit, in particular based on the syringe elements. The pumping unit is configured as an actuator, for example a double syringe pump. In one further embodiment of all aspects, the parallel pulling and/or pushing of the auxiliary medium is controlled by the pumping unit, preferably via the syringe elements. According to one embodiment of all aspects, the syringe pump is a double syringe pump actuator.

In one embodiment of all aspects, the syringe elements are used for pushing and pulling the auxiliary medium, and the syringe elements are not, via their connections to the fluid path system, in contact with any other medium except the auxiliary medium.

In one embodiment of all aspects of the invention, the method further comprises the steps of
opening the fluid path between the or a first fluid reservoir and the first tubing unit and opening the fluid path between the or a second fluid reservoir and the second tubing unit, and
closing the fluid path between the first tubing unit and the mixing element and closing the fluid path between the second tubing unit and the mixing element
   for filling the first tubing unit with the first fluid and filling the second tubing unit with the second fluid.

Furthermore, in one embodiment of all aspects of the invention, the method further comprises the steps of
closing the fluid path between the or a first fluid reservoir and the first tubing unit and closing the fluid path between the or a second fluid reservoir and the second tubing unit, and
opening the fluid path between the first tubing unit and the mixing element and opening the fluid path between the second tubing unit and the mixing element
   for combining the first fluid and the second fluid and for discharging the mixture of the first fluid and the second fluid from the mixing element.

The method as described in any of the embodiments above may be used for aseptic manufacturing of a sterile mixture of a first fluid and a second fluid, or of at least a first fluid and a second fluid. Thus, further fluids may be processed with the inventive arrangement.

In one embodiment of all aspects, the method can be carried out in continuous mode. According to various embodiments of all aspects, a downscaling can be achieved by reducing pumping time. According to various embodiments of all aspects, an upscaling can be achieved by extending pumping time.

Arrangements and techniques described herein realize the controlled mixing of at least two fluid streams under aseptic conditions, preferably by using single use equipment. The systems or setups can be established in an environment which does not fulfill the criteria for aseptic processing. Due to the inventive arrangement, manufacturing does not require an A-class environment, but can be performed in a B-class, C-class or D-class environment. Thus, pharmaceutical manufacturing of several batches, e.g., more than 10 per day, is enabled in an economic way.

### BRIEF DESCRIPTION OF THE DRAWINGS

Exemplary embodiments of the present disclosure are described in detail below with reference to the attached figures. Although the exemplary embodiments of the present disclosure are shown in the drawings, it should be understood that the present disclosure may be implemented in various forms and is not limited by the embodiments described herein.

The drawings are not necessarily to scale. In certain instances, details that are not necessary for an understanding of an embodiment or that render other details difficult to perceive may have been omitted.

The same or equally acting elements are provided with the same reference signs.

FIG. 1A schematically illustrates one embodiment of a mixing system of the present invention comprising a fluid path system and a pumping unit.

FIG. 1B schematically illustrates the mixing system according to FIG. 1A comprising further components.

FIG. 2A illustrates one embodiment of a mixing system of the present invention.

FIG. 2B illustrates the mixing system according to FIG. 2A comprising further components,

FIGS. 3A-3D schematically illustrate states of a mixing process using a mixing system of the present invention.

FIG. 4 schematically illustrates a partial view of one embodiment of a mixing system of the present invention.

FIGS. 5A-5C schematically illustrate states of a mixing process using a mixing system of the present invention.

FIG. 6 shows a flow chart illustrating an exemplary mixing process of the present invention.

### DETAILED DESCRIPTION OF EMBODIMENTS

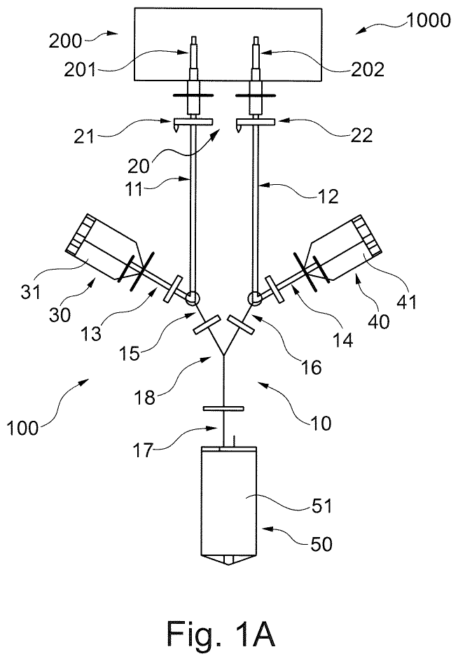

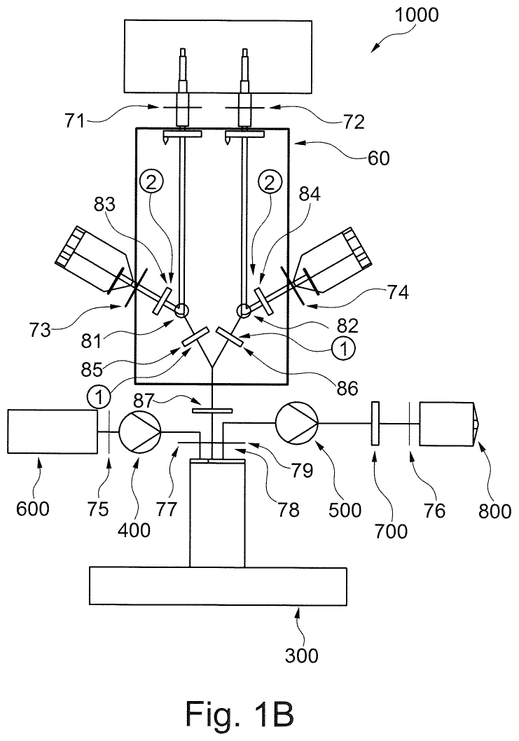

FIG. 1A schematically illustrates an embodiment of a mixing system according to the present invention. The mixing system is configured to provide a mixture of at least two fluids, for example of a first fluid and a second fluid. The first and second fluids may be for example liquids. The mixing system 1000 comprises a sterile fluid path system 100 and a pumping unit 200. FIG. 1B schematically illustrates the mixing system 1000 according to FIG. 1A comprising further components. It should be noted that, for the sake of clarity, the main components of the mixing system are described with regard to FIG. 1A. Connector elements and valve elements are shown in particular in FIG. 1B for the other elements reference is made to FIG. 1A. The reference numerals of the features described with reference to FIGS. 1A and 1B may be present in any one of FIGS. 1A and 1B. In particular, some reference numerals might not be repeated in both FIGS. 1A and 1B. Nevertheless, reference is made to both figures.

In this embodiment, the pumping unit 200 is a syringe pump comprising syringe elements or syringes, in this case, a first syringe element 201 and a second syringe element 202. That is, pumping of the fluids to be mixed is realized by means of the syringe elements, wherein an auxiliary medium, preferably air or gas, is pumped by means of the syringe elements to the fluid path system. Thus, the auxiliary medium applies a pressure to the fluids, so that the fluids are pumped through the fluid path system to provide the mixture.

The syringe elements are preferably operated in a controlled manner by means of a controller. The controller may be provided as part of the pumping unit or can be provided as a separate device. In a preferred embodiment, the syringe pump is an air- or gas-cushioned syringe pump, that is, the syringe elements operate as pumping elements, wherein air or gas is the pumping or auxiliary medium. The auxiliary medium may be a pressure transmitting medium, that is, may be provided as a pressure transmitting medium. Thus, the at least two fluids, for example the two liquids can be forced or conveyed through the mixing system by means of the auxiliary medium.

The fluid path system 100 comprises a tubing set or a fluid path set 10 and a sterile filter unit 20. The sterile filter unit 20, in this embodiment, comprises sterile filter elements or sterile filters, in this case, a first sterile filter element 21 and a second sterile filter element 22. The tubing set 10 can be coupled or connected, that is, is connectable to the pumping unit 200 via the sterile filter unit 20. The sterile filter unit 20 is configured to allow the passage of the auxiliary medium but to block the passage of a fluid which is processed in the mixing system.

In this embodiment, the tubing set 10 and thus, the fluid path system 100, comprises a first tubing unit 11, a second tubing unit 12 and a mixing element 18. The mixing element is configured to mix the at least two fluids; in case of a Y-mixer, mixing is for example carried out by combining the at least two fluids. The sterile fluid path system 100 comprises or is connectable to at least a first fluid reservoir 30 containing a first fluid 31 and a second fluid reservoir 40 containing a second fluid 41. The first fluid 31 and the second fluid 41 may be mixed with the mixing system 1000 so that a mixture 51 of the first fluid 31 and the second fluid 41 can be discharged into a container or bag 50. The tubing set 10 may further comprise connecting tubes, in this embodiment connecting tubes 13, 14, 15, 16, 17, for connecting the fluid reservoirs, the tubing units, the mixing element and the container. In a preferred embodiment, the connecting tubes may be provided as parts of the reservoirs and bags and/or as part of the mixing element. Connecting tubes may also be provided as separate elements.

In this embodiment, each syringe element is connected to a different or separate sterile filter element; furthermore, each filter element is connected to a separate tubing unit. In this case, the first tubing unit 11 is connected to the first sterile filter element 21, and the first sterile filter element 21 is connected to the first syringe element 201. Furthermore, the second tubing unit 12 is connected to the second sterile filter element 22, and the second sterile filter element 22 is connected to the second syringe element 202.

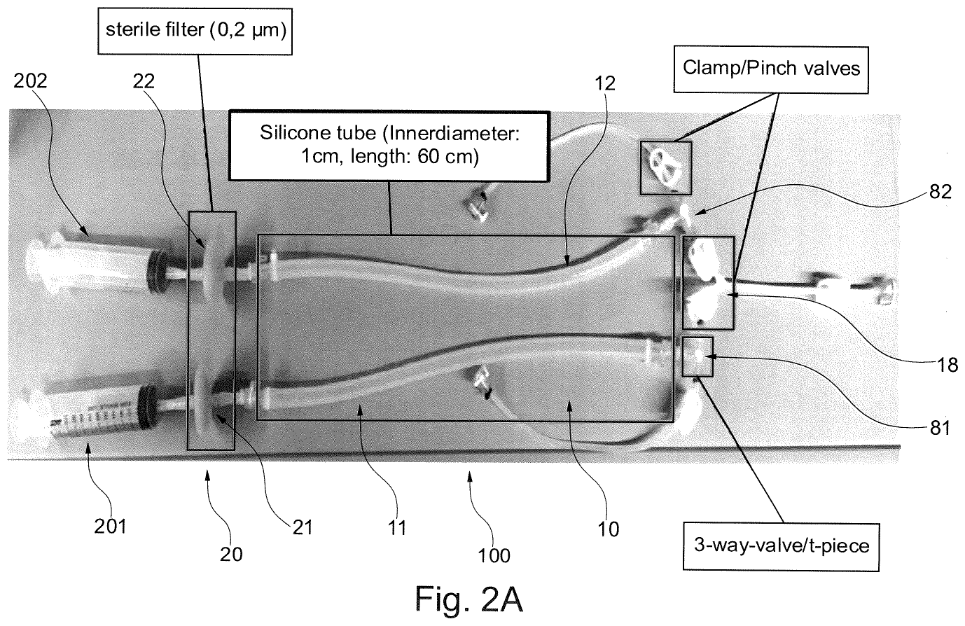

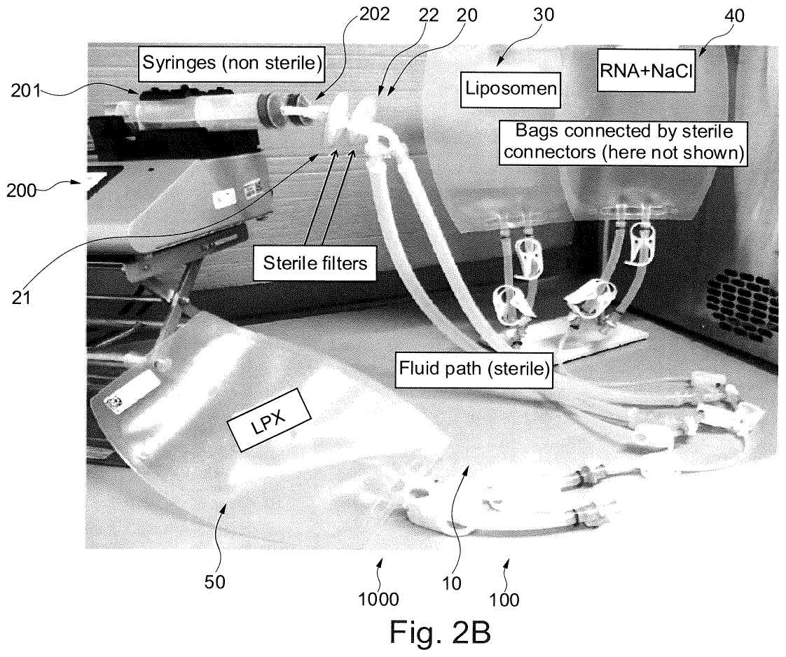

Tubing unit 11 and tubing unit 12 are in this embodiment provided as tube elements or tubes, as also shown in FIGS. 2A and 2B. That is, tubing unit 11 is provided as a tube element 11 and tubing unit 12 is provided as a tube element 12.

In one embodiment of all aspects as described herein, the first tubing unit or first element 11 is connectable to the first sterile filter element 21, and the first sterile filter element 21 is connectable to the first syringe element 201. Furthermore, the second tubing unit or second tube element 12 is connectable to the second sterile filter element 22, and the second sterile filter element 22 is connectable to the second syringe element 202.

In FIG. 1B, further components to the one shown in FIG. 1A are shown. Substantially, only the further components of FIG. 1B with respect to FIG. 1A, are marked by reference numerals. The mixing system 1000 may comprise a rocker device 300, for example a rocker device with integrated scale, which allows for rocking the mixture, for example in bag 50. The mixing system may further comprise one or more pumps 400, 500, for example peristaltic pumps. In this embodiment, pump 400 is configured to supply further substances to the mixture, such as a cryoprotectant or cryoprotectant solution. The substance may be provided in a reservoir 600. The pump 500 may be used to convey the mixture from the container, a first container or bag 50, to a further container, a second container or bag 800, preferably via a filter element 700, for example a 5 μm filter. The container or first container 50 is in this case connected with or connectable to the mixing element 18; the further or second container 800 is connected or connectable to the first container 50.

Sterile connector elements, such as sterile connectors 71, 72 (see FIG. 1B) connect syringe elements 201, 202, respectively, to the sterile filter elements 21, 22. Sterile connectors 71, 72 may be for example Luer-Lock connectors. Further sterile connector elements may be provided, for example connector 73 for connecting reservoir 30 to the tubing set 10 and connector 74 for connecting the reservoir 40 to the tubing set 10.

Further sterile connectors 75, 76, 77, 78, 79 may be provided, for example connector 75 for connecting reservoir 600 to the system, connector 76 for connecting container 800 to the system, connectors 77, 78 and 79 for connecting the container 50 to the system, in particular to connect the container to the pump 400, to the mixing element 18 and/or to the pump 500.

Valve elements, for example stopcocks, clamps, pinch valves and the like are used for controlling the fluid flow from the reservoirs to the containers, for example from the reservoirs to the tubing units and from the tubing units to the mixing element, and preferably also from the mixing element to the container. In this embodiment, a valve element, such as a stopcock 81, for example a three-way stopcock or a T-piece, is arranged between the first fluid reservoir 30 and the first tubing unit 11 and the mixing element 18, respectively. Furthermore, a further valve element, such as a stopcock 82, for example a three-way stopcock or a T-piece, is arranged between the second fluid reservoir 40 and the second tubing unit 12 and the mixing element 18, respectively. Furthermore, further valve elements may be provided, for example clamps, for shutting off the fluid flow. Clamps 83, 84, 85, 86 (see FIG. 1B) and/or clamp 87 can be provided, for example clamp 83 between the first fluid reservoir 30 and the stopcock 81 at connecting tube 13, clamp 84 between the second fluid reservoir 40 and the stopcock 82 at connecting tube 14, clamp 85 between the stopcock 81 and the mixing element 18 at connecting tube 15, clamp 86 between the stopcock 82 and the mixing element 18 at connecting tube 16 and/or clamp 87 between the mixing element 18 and the container 50 at connecting tube 17. The reservoirs and/or the container, in one embodiment, may be provided with corresponding connecting tubes.

It should be noted that the mixing system may be provided with further fluid reservoirs and/or containers.

“Box” 60 may illustrate a sterile bag in which the corresponding components, in particular the components of the tubing set 10, are provided. The components within box 60 may be packaged as an available kit, e.g., for storage, delivery, etc. The components may be used with the sterile bag also during operation. The components may be provided in a pre-assembled manner or may be provided for assembly prior to use, for example, immediately prior to use, where assembling or finalizing the complete fluid path system or the tubing set can be understood as an initial step or steps of any processing performed based on system 100 or 1000.

Assembling of the fluid path system 100 and/or the tubing set 10 can be performed, at least in part or in parts, in a sterile way, optionally under cleanroom conditions.

FIG. 2A illustrates a further embodiment of a mixing system according to the invention. In particular, FIG. 2A illustrates an embodiment of a sterile fluid path system 100 comprising a tubing set 10 and a sterile filter unit 20 with sterile filter elements 21, 22. The tubing set 10 comprises a first tubing unit, in this embodiment a tube element 11, a second tubing unit, in this embodiment a tube element 12 and a mixing element 18. According to an exemplary embodiment, the sterile filter elements 21, 22 may be 0.2 μm filters. According to an exemplary embodiment, tube elements 11, 12 may be silicon tubes, for example with an inner diameter of 1 cm (centimeter) and a length of 60 cm. Other values are contemplated, for example, depending on desired batch sizes, reservoirs, syringes, and/or fluid path configurations as discussed herein. The tube elements 11 and 12 are connected, via the sterile filter elements 21, 22, to syringe elements 201, 202. The first tube element 11 is connected to the first syringe element 201, the second tube element 12 is connected to the second syringe element 202. Valve elements 81 and 82 are implemented as 3-way-valves or t-pieces. Further valve elements are provided which are implemented as clamps or pinch valves.

FIG. 2B illustrates the mixing system according to FIG. 2A comprising further components to the one shown in FIG. 2A. Substantially, only the further components of FIG. 2B with respect to FIG. 2A are marked by reference numerals. The mixing system 1000 comprises the sterile fluid path system 100 as described above and a pumping unit 200. As can be taken in particular from FIG. 2B, the set-up 1000 may be assembled and/or used in an environment, which may be a class B, C or D environment. The pumping unit comprises or is equipped with non-sterile syringe elements 201 and 202 mounted on an actuation device. Bag or reservoir 30 for liposomes, in particular for a liposome colloid, and bag or reservoir 40 for RNA, in particular for an RNA solution, optionally as a NaCl containing solution, are connected by sterile connectors to the tubing set 10. Combining or mixing the liposomes and RNA results in a mixture containing lipoplex particles (LPX). Bag or container 50 for the prepared lipoplex particles (LPX) is connected via a sterile connector to the tubing set 10. Set-up 1000 enables aseptic manufacturing of lipoplex particles, in particular nanoparticles at exactly controlled mixing ratios using the double syringe pumping unit 200 connected by the sterile filter elements 21 and 22 of the sterile filter unit 20 to the fluid path system 100.

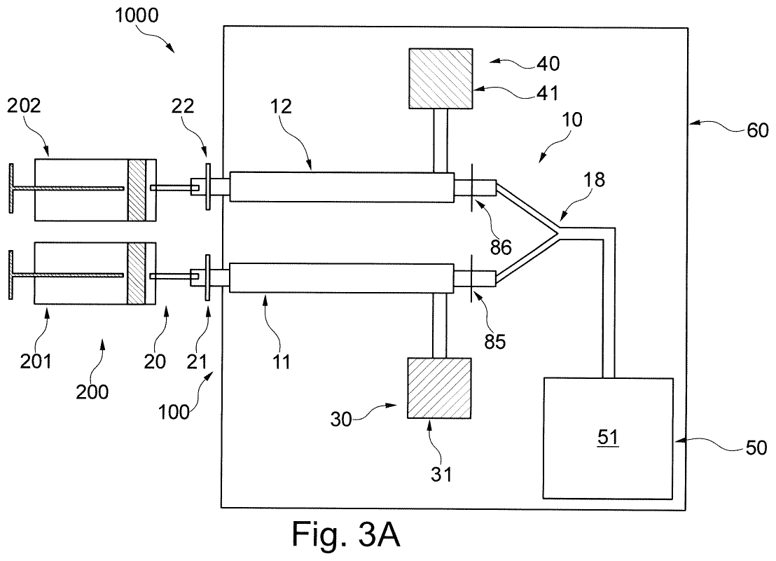

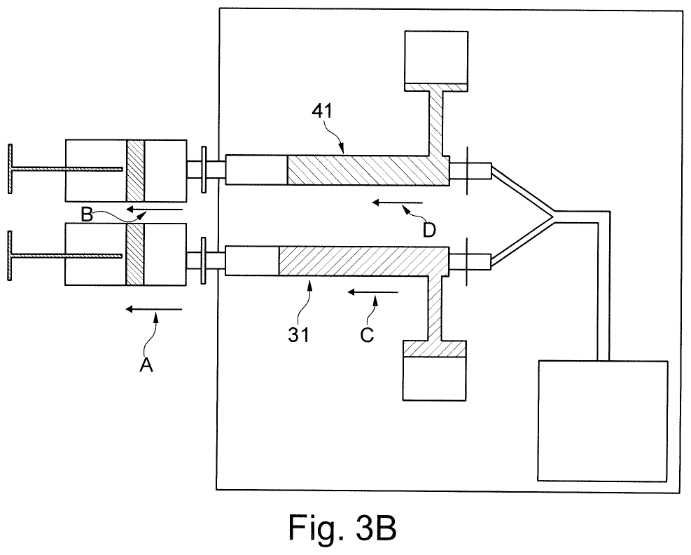

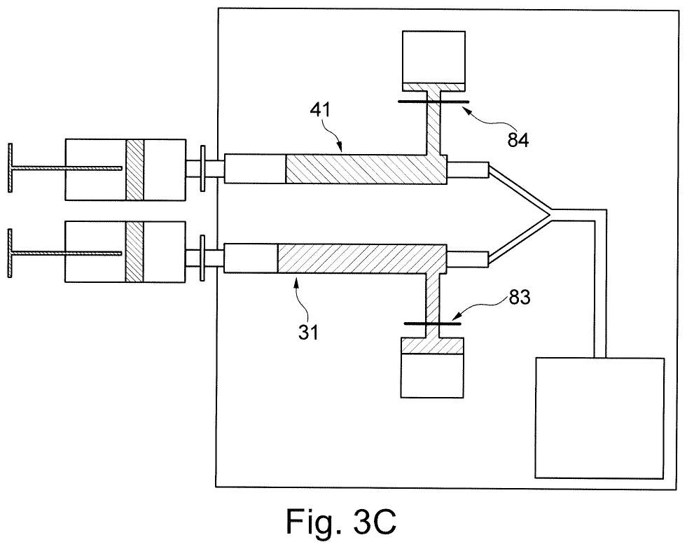

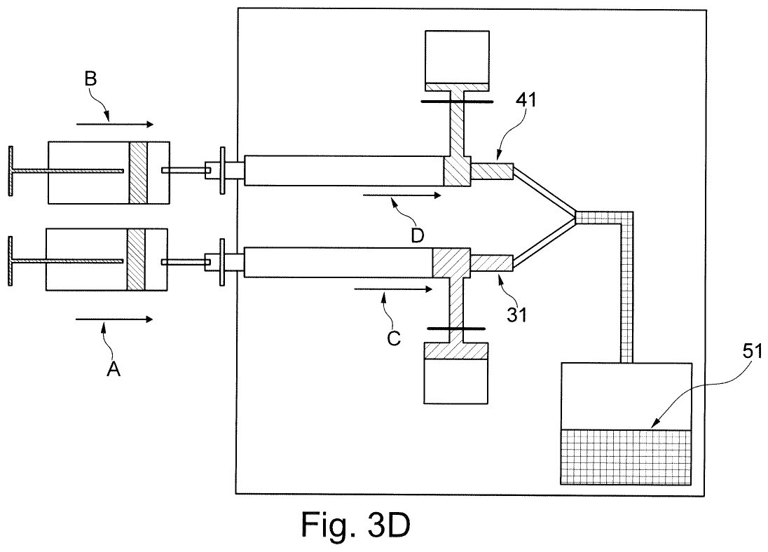

FIGS. 3A-D schematically illustrate an exemplary mixing process based on the use of an arrangement, such as a mixing system 1000. The reference numerals in the description of FIGS. 3A to 3D may be present in any one of FIGS. 3A to 3D. In particular, some reference to numerals might not be repeated throughout the FIGS. 3A to 3D. With reference to FIG. 3A, the mixing system 1000 includes a sterile fluid path system 100 and a pumping unit 200 connected to the fluid path system 100 by means of a sterile filter unit 20. The sterile filter unit 20 comprises, in this case, a first sterile filter element 21 and a second sterile filter element 22. The pumping unit 200 comprises a first syringe element 201 and a second syringe element 202. Thus, each syringe element is connectable to a different or separate sterile filter element. A syringe element may comprise a cylinder and a plunger, moveable in the cylinder, so that air or gas or another suitable auxiliary medium is conveyable via the syringe element. The fluid path system 100, provided, in this embodiment, for example in an aseptic environment, such as a sterile enclosure or bag 60, comprises a tubing set 10. The tubing set 10 comprises at least a first tubing unit or, in this case, a first tube element 11, a second tubing unit or, in this case, a second tube element 12, and a mixing element 18. A first fluid reservoir 30 and a second fluid reservoir 40 are connected by sterile connector elements or connectors (not shown) to the tube elements 11 and 12, respectively. The tube elements 11 and 12 are connected via the mixing element 18 to a product bag or container 50. Valve elements are inserted to allow filling and emptying tube elements 11, 12, as described in the following.

As described above, in this embodiment, the first syringe element 201 is connected to the first tube element 11 via the first sterile filter element 21. Furthermore, the second syringe element 202 is connected to the second tube element 12 via the second sterile filter element 22. The syringe elements are preferably actuated in parallel, so that an optimal and precise mixing process can be carried out.

As an example, bag or reservoir 40 may contain an RNA solution, optionally a NaCl containing RNA solution, as fluid 41, bag or reservoir 30 may contain liposomes as fluid 31, in particular a liposome colloid. The processing is carried out for providing a mixture 51 (see e.g. FIG. 3D) thereof in bag or container 50, that is, RNA lipoplex particles, in particular RNA lipoplex nanoparticles (LPX). During processing, only air or gas may pass through the sterile filter elements 21, 22, but no fluids, in particular no fluids from bags 30 and 40, respectively. It should be noted that “air or gas” is a preferred auxiliary medium, supplied by the pumping unit, for example by the syringe elements, to transport or convey the fluids in the mixing system. Other or further fluids, in particular gaseous substances, such as industrial gas, may also be used for the present mixing process.

FIG. 3A depicts a process step 1. Valve elements, such as clamps 85, 86 between the tube elements 11, 12 and the mixing element 18 are closed. Valve elements between the reservoirs 30, 40 and the tube elements 11, 12 are open. For the sake of clarity, in this figure, only those valve elements are shown which are in a closed state. The plungers of the syringe elements 201, 202 are in an inserted position, that is, the plungers are inserted or substantially inserted in their corresponding cylinders. An inserted position, may be a position in which the plunger is positioned along the fluid path of the cylinder, e.g. a position in which the plunger is at the top dead center of the syringe, e.g. a position in which the plunger is at its nearest or substantially at it nearest to the outlet of the syringe.

The tube elements 11, 12 are “empty”, that is, do not contain fluids to be mixed. The reservoirs 30 and 40 may contain input fluids in an amount at least sufficient to achieve a batch size desired as a result of the processing. Container 50 at this point is “empty”, that is, does not contain a mixture of fluid 31 and fluid 41.

FIG. 3B depicts a process step 2. The plungers of the syringe elements 201, 202 are in an extended position, that is, the plungers are pulled out or substantially or at least partially pulled out from their corresponding cylinders, that is, the syringe elements are pulled open. During pulling the plungers out as indicated by arrows A. B, the cylinders of the syringe elements are filled with air or gas, for example, the “air or gas” from the tube elements 11, 12 (see e.g. FIG. 3A). As a result, the fluids (e.g., liposomes, RNA) are sucked into the tube elements 11, 12, as indicated by arrows C, D. That is, the first tube element 11 is configured for input of the first fluid 31, and the second tube element 12 is configured for input of the second fluid 41. In other words, the first tube element 11 is configured to receive a fluid, in this case, the first fluid 31, and the second tube element 12 is configured to receive a fluid, in this case, the second fluid 41. With filling of the tube elements, the corresponding reservoirs are emptied, at least partially, preferably completely.

In one embodiment the tube volume of each of the tube elements 11, 12 corresponds or at least substantially corresponds to the volume of the corresponding syringe element 201, 202 respectively. That is, the volume of the tube element 11 may correspond at least substantially with the volume of the syringe element 201 and/or the volume of the tube element 12 may correspond at least substantially with the volume of the syringe element 202. In one embodiment, each of the tube elements 11, 12 is long enough or comprises a volume so that the entire fluid of for example the corresponding fluid reservoir can be received within the tube element. In other words, the tube elements comprise preferably a storage capacity such that a predetermined amount of a respective fluid is storable within the first tubing unit and/or the second tubing unit, wherein, optionally, the predetermined amount is an amount which can be mixed with one pumping stroke of the syringe elements. In one embodiment, the reservoirs 30 and 40 initially contain exactly the amount of fluid which can be processed within one processing cycle, in particular with one pumping stroke of the syringe elements.

FIG. 3C depicts a process step 3, where the valve elements are switched. Valve elements 83, 84 between the reservoirs 30, 40 and the tube elements 11, 12 are closed. Valve elements between the tube elements 11, 12 and the mixing element 18 are open. Also in this figure, only the valve elements in a closed position are shown.

FIG. 3D depicts a process step 4. The plungers of the syringe elements 201, 202 are again in the inserted position, which means, the plungers are inserted or substantially inserted in their corresponding cylinders. During pushing the plungers into the cylinders, as indicated by the arrows A, B, air or gas within the syringe elements 201, 202 is pumped into the tube elements 11, 12 via the sterile filter elements 21, 22 and applies a pressure on the fluids in the tube elements. As a result, the fluids are pumped or conveyed from the tube elements to the mixing element 18 and to the container 50, as indicated by arrows C, D. Due to the sterile filter elements 21, 22, only sterile air or gas is supplied to the tube elements 11, 12. The mixture 51 of the first fluid 31 and the second fluid 41 is collected in the container 50. The mixture 51 may be for example LPX.

In order to allow for a precise and efficient mixing process, the syringes are preferably activated in parallel. That is, the plunger of the syringe element 201 and the plunger of the syringe element 202 are pulled out, e.g. pulled towards the end of the cylinder most distant to the outlet, from their corresponding cylinders and/or pushed into their cylinders in parallel.

FIGS. 3A-D illustrate batch manufacturing based on a single pull-push operation. i.e. fluids, in particular liquids, are completely or substantially completely sucked from fluid reservoirs into the fluid path system in a single pull operation, and are then, in a single push operation, pushed via the mixing element into the batch receiving container. It is to be understood that according to other examples, batch manufacturing may also involve two or more, i.e. repeated pull-push operations, where any particular pull-push operation may, for example, suck and mix only parts of the liquids contained in the fluid reservoirs. Fluid path systems may be employed for varying batch sizes.

Optional further processing steps may comprise, that a cryoprotectant solution and/or another substance be added from a reservoir which is connected to the fluid path system via a sterile connector. The cryoprotectant may be pumped via a pump, preferably via a peristaltic pump, to the product bag or container. A further optional step may comprise rocking the mixture or solution with or without the cryoprotectant and/or other substances in the product container for, e.g., 10 minutes. A further optional step may comprise that the mixture or solution with or without the cryoprotectant and/or other substances be filtered by a filter element, for example a 5 μm filter element and pumped to a further container via a further pump, preferably via a peristaltic pump. As a result of the aseptic manufacturing process, a desired amount of ready to use mixture, for example LPX, is available in the further container or product bag.

While the processing or manufacturing steps have been described in a particular sequence for ease of explanation and understanding, it should be noted that various steps and (partial) sequences of steps can be re-arranged. For example, the connection of bags, syringes, etc. can be performed in varying order. Furthermore, it should be noted that the steps and (partial) sequences of steps can be repeatedly performed. For example, the sequence of pulling the air or gas out of and pushing the air or gas into the fluid path system can be performed repeatedly, for example to achieve a desired batch size/amount of fluid in containers 50 or 800. Preferably, one pull-/push operation is used for manufacturing the mixture 51.

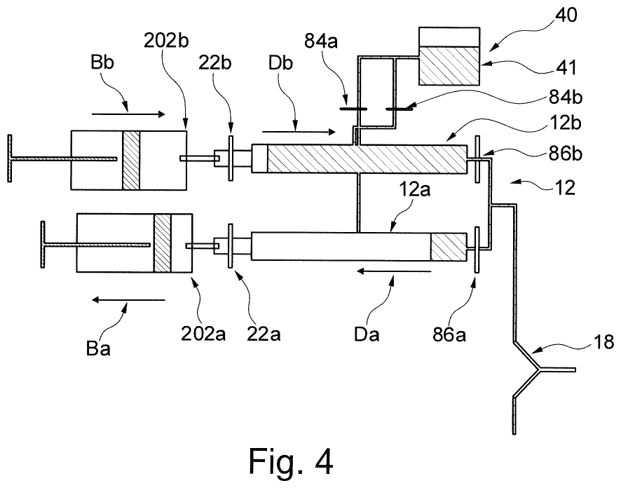

FIG. 4 schematically illustrates a partial view of an embodiment of a mixing system of the present invention enabling continuous pumping. One portion, e.g. one half, of the setup is shown in FIG. 4. Each tubing unit may comprise at least two tube elements which are both connectable to one or more respective fluid reservoirs. In this embodiment, both tube elements are connected to fluid reservoir 40. FIG. 4 shows tubing unit 12 comprising a first tube element 12a and a second tube element 12b. Tubing unit 11 is not shown. Each tube element 12a, 12b is connectable to a syringe element or a syringe pump of a pumping unit, wherein each tube element is connectable to a different syringe element. In this embodiment, tube element 12a is connected to syringe element 202a and tube element 12b is connected to syringe element 202b. With such an arrangement and a corresponding control of the pumping procedure, a continuous flow of the fluids without any interruption can be provided. A similar arrangement is provided for tubing unit 11 (not shown). The tubing units may comprise more than two tube elements, for example three, four or more tube elements.

Valve elements, such as inlet valves or clamps 84a, 84b are provided at connections or connecting tubes from bag 40 to tube elements 12a, 12b. Further valve elements, such as outlet valves or clamps 86a, 86b are provided at connections or connecting tubes from tube elements 12a, 12b to Y-mixer or mixing element 18 for mixing with fluid from the other portion, e.g. half, (not shown).

Each tube element 12a, 12b, and also the tube elements of the other half, is connected to the corresponding syringe element via a sterile filter element. FIG. 4 shows sterile filter elements 22a and 22b.

In this embodiment, four tube elements are provided (only two are shown); thus, also four syringe elements are provided. In a preferred embodiment, pumping is controlled such that the syringe elements of each portion, e.g. half, run in opposite directions, in particular substantially in opposite directions, however, in such a manner that for example the bottom dead center of one of the syringe elements and the top dead center of the other of the syringe elements are reached at different times. In any way, if a continuous flow of a corresponding fluid to the mixing element is needed, the syringe elements have to be controlled so that an interruption of the flow in direction to the mixing element is avoided.

At the particular stage as shown in FIG. 4, the plunger of syringe element 202b is inserted or pushed into the cylinder of the syringe element, which means air or gas in the cylinder is pushed to tube element 12b, as indicated by arrow Bb. This leads to fluid 41 from tube element 12b being pushed via open outlet valve 86b towards Y-mixer 18 as indicated by arrow Db, while inlet valve 84b is closed. At the same time, the plunger of syringe element 202a is pulled out from the cylinder, which means air or gas from tube element 12a is sucked into the cylinder of the syringe element, as indicated by arrow Ba. This leads to fluid 41 from bag 40 entering tube 12a via open inlet valve 84a, as indicated by arrow Da, while outlet valve 86a is closed. Similar procedure is carried out with the tube element 11 (not shown). Due to the filter elements. shown are filter elements 22a, 22b, only sterile air or gas is supplied to the tube elements of the tubing units.

Proper control of syringe pumps and/or inlet and outlet valves results in a continuous flow of the fluids towards Y-mixer or mixing element 18. That is, also in case of an arrangement for continuous flow, the two portions, e.g. the two halves, are preferably coordinated, such that a precise and accurate mixture process is possible; the portion, e.g. halves, are thus preferably actuated in parallel.

In an alternative embodiment, the tube elements of each portion, e.g. half, may be connected to different reservoirs of corresponding fluids.

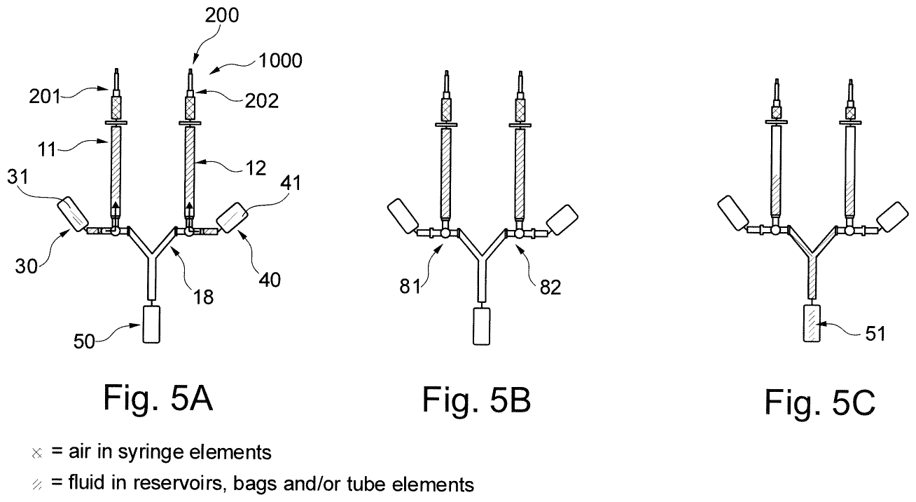

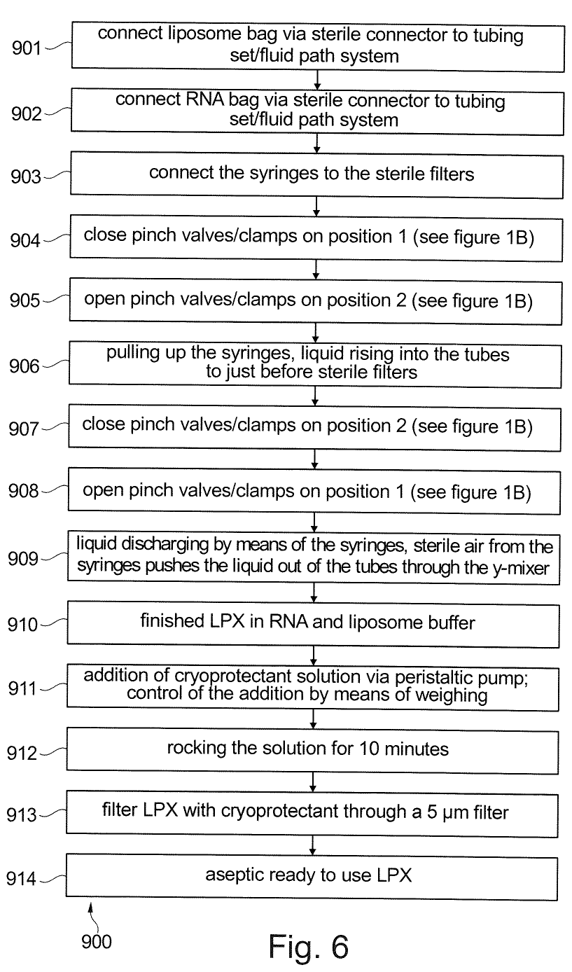

FIGS. 5A-5C schematically illustrate in a simplified representation the states of a mixing process using a mixing system 1000 of the present invention. The reference numerals in the description of FIGS. 5A to 5C may be present in any one of FIGS. 5A to 5C. In particular, some reference numerals might not be repeated throughout the FIGS. 5A to 5C.

In FIG. 5A, the first fluid 31 of first fluid reservoir 30 and the second fluid 41 of the second fluid reservoir 40 are pumped via the pumping unit 200 into the first tube element 11 and the second tube element 12, respectively. In a preferred embodiment, as shown in FIG. 5B, the fluids 31, 41 of the reservoirs 30, 40 are now completely stored in the tube elements 11, 12. After switching corresponding valves, only stopcocks 81, 82 are shown, the fluids within the first tube element and the second tube element are pumped via the pumping unit 200 to the mixing element 18 and after combining or mixing the first fluid 31 and the second fluid 41, the mixture 51 of the first fluid 31 and the second fluid 41 is pumped to container 50, see FIG. 5C. Pumping is realized by, for example, air or gas, transported by means of the syringe elements 201, 202. In the figures the first fluid and second fluid are shown through diagonal dashes. The air or gas in the syringe elements is shown through crosses.

FIG. 6 shows a flow chart illustrating an exemplary mixing process 900 for aseptic manufacturing of a mixture of a first fluid and a second fluid, such as a sterile LPX of liposomes as a first fluid and RNA as a second fluid. It should be noted that also RNA can be indicated as the first fluid and liposomes can be indicated as the second fluid. The components of the mixing system 1000 as described for example with FIGS. 1A and 1B or 2A and 2B may be used.

Method step (not shown): Providing a set of components or pieces with quantities such as illustrated and discussed for example with reference to FIGS. 1A and 1B or 2A and 2B.

In further method steps, the components have to be connected to each other. With the connection of the components a fluid path system and mixing system as illustrated in FIGS. 1A and 1B, 2A and 2B may be obtained. Connection may comprise connecting sterile components such as tubing, valves, sterile filter elements. Y-mixer or mixing element, wherein sterile conditions are preserved by using sterile connectors and/or operating in an aseptic environment. Providing the fluid path system may also comprise connecting the bags or reservoirs containing the fluids to be mixed to the tubing set 10. Providing the fluid path system may also comprise connecting the bag(s) or container(s) for receiving the mixture of the fluids to the tubing set 10. Providing the fluid path system may also comprise inserting valves, such as clamps, to allow filling and emptying the tubing units, for example tube elements. Providing the fluid path system may also comprise connecting the sterile filter unit to tubing set 10. Providing the mixing system may also comprise connecting the pumping unit to the fluid path system. Connection of the components may be performed in a preparatory step prior to, e.g. immediately prior to the subsequent manufacturing of the desired mixture.

Method step 901: Connecting at least a first fluid reservoir, for example fluid reservoir 30 with a first fluid 31, for example a liposome bag, via a sterile connector to the fluid path system 100 or tubing set 10. In an alternative embodiment, the fluid reservoir may be part of the fluid path system and is already connected to the tubing set 10.

Method step 902: Connecting at least a second fluid reservoir, for example fluid reservoir 40 with a second fluid 41, for example an RNA bag, via a sterile connector to the fluid path system 100 or the tubing set 10. In an alternative embodiment, the fluid reservoir may be part of the fluid path system and is already connected to the tubing set 10.

Method step (not shown): Connecting a least one bag for the manufactured mixture 51, for example container 50, via a sterile connector to the fluid path system 100 or the tubing set 10. In an alternative embodiment, the bag or container may be part of the fluid path system and is already connected to the tubing set 10.

Method step 903: Connecting the pumping unit 200 to the sterile filter unit 20 and thus, to the tubing units. The sterile fluid paths, for example tubing set 10 are/is coupled to the pumping unit via the sterile filter unit, for example via the sterile filter elements for each syringe element and each tube element. The first syringe element 201 may be connectable to the first tubing unit or tube element 11 via the first sterile filter element 21; the second syringe element 202 may be connectable to the second tubing unit or tube element 12 via the second sterile filter element 22.

In one embodiment, the syringe elements may be provided in the pumping unit, such as a syringe pump, installable in the pumping unit. In another embodiment, the syringe elements may be part of the fluid path system.

It is to be understood that steps 901 to 903 can be performed in any order, including being performed in parallel.

In an alternative embodiment, the fluid path system may be provided as a connected set.

Method step 904: Adjusting the valve elements, such as for example pinch valves or clamps. In accordance with FIG. 1B, closing valve elements 85 and 86 at position 1.

Method step 905: Adjusting the valve elements, such as for example pinch valves or clamps. In accordance with FIG. 1B, opening valve elements 83 and 84 at position 2.

It is to be understood that steps 904 and 905 can be performed in any order, including being performed in parallel.

Method step 906: Activating the pumping unit 200, in this embodiment, the syringe elements 201, 202, in particular in parallel, by pulling out, e.g. pulling towards the end of the cylinder being most distant to the outlet, e.g. pulling towards the bottom dead center, the respective syringe plungers, that is, by pulling out the syringe elements, so that the first fluid 31 is drawn into the first tube element 11 and that the second fluid 41 is drawn into the second tube element 12, by means of the sterile auxiliary medium, which is preferably air or gas. That is, pumping is carried out preferably through syringe elements which operate with air or gas. In other words, the syringe elements of the pumping unit are pulled open or pulled up, thus fluids, for example liposomes, RNA, are sucked into the tubing units or tube elements of the tubing set or may rise inside the tube elements, preferably just before the corresponding sterile filter element.

Each of the tubing units or tube elements preferably comprises a storage capacity which allows for input of the complete fluids to be mixed. In a preferred embodiment, mixing is performed with a mixing ratio of 1:1 of the fluids from the reservoirs. In other embodiments, the system operates with other mixing ratios, where the volumes of the syringe elements and/or tubing units or tube elements may be adapted accordingly. In one embodiment, a controller is provided for controlling the activation of the pumping unit.

Method step 907: Adjusting the valve elements, such as for example pinch valves or clamps. In accordance with FIG. 1B, closing valve elements 83 and 84 at position 2.

Method step 908: Adjusting the valve elements, such as for example pinch valves or clamps. In accordance with FIG. 1B, opening valve elements 85 and 86 at position 1.

These operations may be performed in any order, including being performed in parallel.

In embodiments where the valve elements are provided as three-way valves or T-pieces, a corresponding adjustment may be required to prevent or allow fluid flow in the required direction.

Method step 909: Activating the pumping unit 200, in this embodiment, the syringe elements 201, 202, in particular in parallel, by activating the respective syringe plungers, so that the first fluid 31 and the second fluid 41 are discharged or pushed out from the first tubing unit or tube element 11 and the second tubing unit or tube element 12 through the mixing element 18 and into the bag or container 50 for the mixture 51 of the first fluid 31 and the second fluid 41, for example lipoplex LPX. Pushing may be performed by means of the sterile auxiliary medium, which is preferably air or gas. In other words, the two (air- or gas-filled) syringes are emptied, preferably in parallel, the air or gas passes through the sterile filters and applies pressure on the fluids in the tubing units, which pushes the fluids in parallel through the mixing element 18. The sterile filter unit is configured to only allow the passage of an auxiliary medium, for example air or gas, and to prevent the passage of the fluids to be mixed. Thus, only a sterile auxiliary medium, for example sterile air or gas, is conveyed to the tubing units. The pressure transferred via the auxiliary medium to the tubing units allows for combining, that is, mixing of the at least first and second fluids.

Method step 910: Providing the finished LPX in RNA and liposome buffer, in particular with the desired batch size in output bag or container 50. Mixing can be carried out with one or more pumping cycles.

Method step 911: Optionally, adding one or more further substances, such as a cryoprotectant solution, to the mixture 51, preferably via a peristaltic pump. A control of the addition of the further substance may be performed by means of weighing.

Method step 912: Optionally, rocking, stirring or otherwise treating the mixture with or without the further substance(s), preferably for 10 minutes. These additional steps are for example provided to achieve homogeneity of the mixture.

Method step 913: Optionally, filtering the mixture with or without the further substance(s), preferably through a 5 μm filter element.

Method step 914: Then, the ready to use aseptic mixture is obtained, for example ready to use LPX.

Method step (not shown): Optionally, re-filling the mixture with or without the further substance(s) to achieve desired batch sizes, etc.

Opening of a valve element allows the fluid and the mixture, which is also a fluid, to flow through the fluid path; closing of a valve element blocks the flow of fluid through the fluid path. That is, an open valve element provides an open fluid path, a closed valve element blocks the fluid path. For example “opening” or “closing” a clamp is to be understood as “opening” or “closing” a passage of a fluid path.

While the method steps have been described in a particular sequence for ease of explanation and understanding, it should be noted that various steps and (partial) sequences of steps may also be re-arranged. For example, the connection of bags, syringe elements, etc. can be performed in varying order. It should be noted that steps and (partial) sequences of steps can be repeatedly performed. For example, the sequence of pulling the air or gas out of and pushing the air or gas into the fluid path system can be performed repeatedly, for example to achieve a desired batch size/amount of mixture in the product container.

It is preferred that for strict temporal control of a mixing ratio, the pumping unit may preform the pushing and/or pulling operations as strictly as possible in parallel and with, for example, constant pushing and/or pulling operations for the required time period. Automatic control of the operations may be preferred, however, also a manual control, for example a manual operation of the syringe elements, may be sufficient in specific applications. The pushing and/or pulling operations may be controlled in order that desired amounts of the mixture is pushed into bag 50. The pumping unit is preferably programmable to consider the corresponding amounts of air or gas or auxiliary medium, speed and/or number of pumping cycles, necessary for a corresponding mixing cycle, in particular depending on a pre-defined batch size.

The inventive arrangement and mixing procedure allow for an aseptic manufacturing of a mixture, for an intrinsically pulsation-free operation, and for a decrease or avoidance of shear forces compared to operations with peristaltic pumps. Furthermore, the arrangement and procedure are suitable for very small batch sizes. As described herein, the arrangement may be operated in continuous mode; unsealing is possible just by extending the pumping time, downscaling can be achieved by reducing the pumping time. Arrangements and procedures or processes are economic, since the set-tup does not use complex components. In particular, single use components may be used which are simple in design. Arrangements and procedures are intrinsically safe and can be controlled very easy and accurate at any time, in particular the pumping and/or mixing conditions.

The fluid path system and/or tubing set can be provided in packages or kits. Each package or kit may comprise one or more of the systems and/or sets. That is, a package may comprise a plurality of identical systems and/or sets. A package or kit may also comprise corresponding connector elements and/or valve elements. The components of the fluid path system and/or tubing set may be provided as sterile components and a package or kit may comprise a plurality of components. In an embodiment, components may be individually packaged.

The fluid path system or mixing system and use thereof, manufacturing method and embodiments described herein, can be employed in various environments in the medical and veterinary fields, but also in other fields such as, for example, laboratory areas.

Although the present disclosure is described in detail herein, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

The elements of the present disclosure as described herein are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present. i.e. for the purpose of this embodiment “comprising” is to be understood as having the meaning of “consisting of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety.

### Definitions

In the following, definitions will be provided which apply to all aspects and embodiments of the present disclosure. The following terms have the following meanings unless otherwise indicated. Any undefined terms have their art recognized meanings.

Terminology such as “pharma grade” or “pharmaceutical manufacturing” refers to manufacturing processes and equipment that are used to produce or manufacture pharmaceutical products, for example drugs and other medications, such that these drugs meet predefined standards of approval.

The term of “Good Manufacturing Practice”, “GMP”, refers to guidelines for quality assurance with respect to the manufacture of pharmaceutical products.

The term “batch” as used herein refers to a defined quantity of a product.

The terms “A, B, C or D class environment” refer to an environment with pre-defined conditions such as the particle number, temperature and humidity, for example. For the manufacturing of sterile products, grade or class A environment is required, which provides strict aseptic conditions. Class B, C or D environments may be sufficient for less critical stages in a manufacturing process.

The terms “combining fluids in the mixing element” or “the combination of the fluids in the mixing element” as used herein means that a mixture of the fluids to be mixed, for example of the first fluid and the second fluid, is provided. In the simplest form, combining of fluids means mixing the fluids by bringing the fluids together.

The terms “syringe”, “syringe element” or “syringe pump” and the like are to be understood as referring to a device which is capable to administer a certain amount of a fluid, in the present case in particular air or gas.

In the context of the present disclosure, the term “particle” relates to a structured entity formed by molecules or molecule complexes. In one embodiment, the term “particle” relates to a micro- or nano-sized structure, such as a micro- or nano-sized compact structure.

In the context of the present disclosure, the term “RNA lipoplex particle”, “lipoplex particle”, “lipoplex” or “LPX” relates to a particle that comprises lipids, preferably cationic lipids, and RNA (most preferably mRNA). Electrostatic interactions between positively charged lipids and negatively charged phosphate groups of the RNA results in complexation and spontaneous formation of RNA lipoplex particles. Cationic lipids allow electrostatic interactions with the RNA, while co-lipids also comprised in the lipoplex particle contribute to a reduced cytotoxicity and can induce the destabilization of the endosomal membrane that favors mRNA transfer from the endosome to the cytosol. DOPE (1,2-Dioleoyl-sn-glycero-3-phosphoethanolamine) is preferably comprised in the lipoplex particles used in the invention because it has the propensity to induce favorable supramolecular changes (from lamellar to inverted hexagonal supramolecular assemblies), thus promoting membrane fusion in a mild acid environment typical of the endosome lumen. Thus, RNA lipoplex particles preferably comprise a cationic lipid, such as DOTMA, and additional lipids, such as DOPE. In one embodiment, an RNA lipoplex particle is a nanoparticle.

As used in the present disclosure, “nanoparticle” refers to a particle comprising RNA and at least one cationic lipid and having an average diameter suitable for intravenous administration, whereby the average diameter is preferably, between 10 and 990 nm, such as between 15 and 900 nm, between 20 and 800 nm, between 30 and 700 nm, between 40 and 600 nm, between 50 and 500 nm, or between 300 nm and 500 nm.

The term “average diameter” refers to the mean hydrodynamic diameter of particles as measured by dynamic light scattering (DLS) with data analysis using the so-called cumulant algorithm, which provides as results the so-called Z_{average} with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

RNA lipoplex particles described herein may have an average diameter that in one embodiment ranges from about 200 nm to about 1000 nm, from about 200 nm to about 800 nm, from about 250 to about 700 nm, from about 400 to about 600 un, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nr, about 350 nm, about 375 nm, about 400 nm, about 425 nm, about 450 nm, about 475 nm, about 500 nm, about 525 nm, about 550 nm, about 575 nm, about 600 nm, about 625 rm, about 650 nm, about 700 run, about 725 nm, about 750 run, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 rim. In an exemplary embodiment, the RNA lipoplex particles have an average diameter of about 400 nm.

Generally, the RNA lipoplex particles described herein are obtainable by adding RNA to a colloidal liposome dispersion. The colloidal liposome dispersion is obtainable by ethanol injection technique. The term “ethanol injection technique” refers to a process, in which an ethanol solution comprising lipids is rapidly injected into an aqueous solution through a needle. This action disperses the lipids throughout the solution and promotes lipid structure formation, for example lipid vesicle formation such as liposome formation. Using the ethanol injection technique, such colloidal liposome dispersion is, in one embodiment, formed as follows: an ethanol solution comprising lipids, such as cationic lipids like DOTMA and additional lipids such as DOPE, is injected into an aqueous solution under stirring. In one embodiment, the RNA lipoplex particles described herein are obtainable without a step of extrusion.

The term “extruding” or “extrusion” refers to the creation of particles having a fixed, cross-sectional profile. In particular, it refers to the downsizing of a particle, whereby the particle is forced through filters with defined pores.

In one embodiment, the lipid solutions, liposomes and RNA lipoplex particles described herein include a cationic lipid. As used herein, a “cationic lipid” refers to a lipid having a net positive charge. Cationic lipids bind negatively charged RNA by electrostatic interaction to the lipid matrix. Generally, cationic lipids possess a lipophilic moiety, such as a sterol, an acyl or diacyl chain, and the head group of the lipid typically carries the positive charge. Examples of cationic lipids include, but are not limited to 1,2-di-O-octadecenyl-3-trimethylammonium propane (DOTMA), dimethyldioctadecylammonium (DDAB); 1,2-dioleoyl-3-trimethylammonium propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioicyloxypropyl-3-dimethyl-hydroxyethyl ammonium bromide (DORIE), and 2,3-dioleoyloxy-N-[2(spermine carboxamide)ethyl]-N,N-dimethyl-1-propanamium trifluoroacetate (DOSPA). Preferred are as DOTMA, DOTAP, DODAC, and DOSPA. In specific embodiments, the at least one cationic lipid is DOTMA and/or DOTAP.

An additional lipid may be incorporated to adjust the overall positive to negative charge ratio and physical stability of the RNA lipoplex particles. In certain embodiments, the additional lipid is a neutral lipid. As used herein, a “neutral lipid” refers to a lipid having a net charge of zero. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, cholesterol, and cerebroside. In specific embodiments, the second lipid is DOPE, cholesterol and/or DOPC.

In certain embodiments, the RNA lipoplex particles include both a cationic lipid and an additional lipid. In an exemplary embodiment, the cationic lipid is DOTMA and the additional lipid is DOPE. Without wishing to be bound by theory, the amount of the at least one cationic lipid compared to the amount of the at least one additional lipid may affect certain RNA lipoplex particle characteristics, such as charge, particle size, stability, tissue selectivity, and bioactivity of the RNA. Accordingly, in some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a β-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as modified ribonucleotides or deoxynucleotides. Examples of modified ribonucleotides include, without limitation, 5-methylcytidine and pseudouridine.

In certain embodiments of the present disclosure, the RNA is messenger RNA (mRNA) that relates to an RNA transcript which encodes a peptide or protein. As established in the art, mRNA generally contains a 5′ untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription. The cDNA may be obtained by reverse transcription of RNA.

In some embodiments, the RNA according to the present disclosure comprises a 5′-cap. In one embodiment, the RNA of the present disclosure does not have uncapped 5′-triphosphates. In one embodiment, the RNA may be modified by a 5′-cap analog. The term “5′-cap” refers to a structure found on the 5′-end of an mRNA molecule and generally consists of a guanosine nucleotide connected to the mRNA via a 5′ to 5′ triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes.

In some embodiments, the RNA according to the present disclosure comprises a 3′-poly(A) sequence. The term “poly(A) sequence” relates to a sequence of adenyl (A) residues which typically is located at the 3′-end of an RNA molecule. According to the disclosure, in one embodiment, a poly(A) sequence comprises at least about 20, at least about 40, at least about 80, or at least about 100, and up to about 500, up to about 400, up to about 300, up to about 200, or up to about 150 A nucleotides, and in particular about 120 A nucleotides.

According to the disclosure, the term “RNA encodes” means that the RNA, if present in the appropriate environment, such as within cells of a target tissue, can direct the assembly of amino acids to produce the peptide or protein it encodes during the process of translation. In one embodiment, RNA is able to interact with the cellular translation machinery allowing translation of the peptide or protein. A cell may produce the encoded peptide or protein intracellularly (e.g. in the cytoplasm and/or in the nucleus), may secrete the encoded peptide or protein, or may produce it on the surface.

According to the disclosure, the term “peptide” comprises oligo- and polypeptides and refers to substances which comprise about two or more, about 3 or more, about 4 or more, about 6 or more, about 5 or more, about 10 or more, about 13 or more, about 16 or more, about 20 or more, and up to about 50, about 100 or about 150, consecutive amino acids linked to one another via peptide bonds. The term “protein” refers to large peptides, in particular peptides having at least about 151 amino acids, but the terms “peptide” and “protein” are used herein usually as synonyms.

According to the disclosure, the term “cryoprotectant” relates to a substance that is added to a formulation in order to protect the active ingredients during freezing stages.

The term “polydispersity index” or “PDI” is used herein as a measure of the size distribution of an ensemble of particles, e.g., nanoparticles. The polydispersity index is calculated based on dynamic light scattering measurements by the so-called cumulant analysis.

The term “PCS” means “photon correlation spectroscopy” with respect to the term “photon correlation spectroscopy (PCS) measurements”.

It is to be understood that the terminology used herein is for the purpose of describing particular embodiments only and is not intended to limit the scope of the present invention which will be limited only by the appended claims. Unless defined otherwise, all technical and scientific terms used herein have the same meanings as commonly understood by one of ordinary skill in the art.

### EXAMPLE

In one example, the following elements may be used in a mixing system of the present invention:

TABLE 1

| Component | Description | Quantity |
|---|---|---|
| Pumping unit | Syringe pump, Kd Scientific, Legato210, C200401 | 1 |
| Syringe element | Bionic; 100 mL JMS Disposable Syringe; Partno. | 2 |
| | 551721052 | |
| Connector element | ⅛″ Female Luer for ¼″ 28 Bullehead Mounting | 2 |
| Sterile filter unit | Sartorius stedim Midisart; sterile filter 0.2 μm, | 2 |
| Sterile filter element | 2000; Typ: 17805; Lot: 80275113 | |
| Tubing unit, | Silicone tube; inner diameter 1 cm, length 60 cm | 2 |
| Tube element | | |
| Valve element | Nordson Medical, Stopcock, 3-way, Female Luer | 2 |
| | to Female Luer to Male; Ref: FCFFMR-003; Lot: | |
| | 162096-*-* | |
| Connection tube, e.g. | Silicone Altimex, silicone tube, inner diameter 2.4 | 2 |
| tube of reservoirs | mm, length 30 cm; Lot: 83739; PartNo. 1-SA P60 | |
| | 24-16-56 | |
| Fluid reservoir | Thermofischer; 1 liter bag; 1487 Lot: ECH254347 | 2 |
| Valve element | IVAC Set F7779F_18280; Ref: Set 4 Overpack: 1; | 4 |
| | Lot: K81NBEG | |
| Mixing element | Y-Type 2.4 mm, Nordson Company, from IVAC | 1 |
| | Set Z8472X_18617; Set 2 Overpack 1 | |
| Connector | Combifix Adapter; B. Braun; Ref: 5206642; Lot: | 2 |
| | 15G15A8171 | |
| Connector | MPC Male connectors | 8 |
| Connector | MPC Female connectors | 8 |

As an implementation of the pumping unit, a double syringe pump actuator may be utilized. Syringe elements may, for example, be implemented as 100 ml (milliliters) disposable syringes. Sterile filter elements may be provided as 0.2 μm (micrometers) (air or gas) filters. Tube elements may be provided as pharma grade silicone tubes with an inner diameter of 1 cm and a length of 60 cm.

Experimental Results from LPX Manufacturing

Analysis of an exemplary manufactured LPX batch manufactured with systems and techniques of the present invention:

| Sample | diameter/nm | PDI |
|---|---|---|
| LPX only (w/o) | 352.9 | 0.194 |
| Cryoprotectant) | | |
| final LPX | 401 | 0.141 |

For PCS measurements the sample was diluted with 0.9% NaCl to a RNA concentration of 0.01 mg/mL.

| RNA content | Abs | average | mg/ml |
|---|---|---|---|
| final LPX | 0.254 | 0.249 | 0.020 |
| final LPX | 0.248 | | |
| final LPX | 0.244 | | |

Measured RNA concentration of the prepared RNA-LPX (target 0.020 mg/mL).

### LIST OF REFERENCE NUMBERS

10 tubing set
11 first tubing unit, first tube element
12 second tubing unit, second tube element
12a first tube
12b second tube
13 connecting tube
14 connecting tube
15 connecting tube
16 connecting tube
17 connecting tube
18 mixing element
20 sterile filter unit
21 first sterile filter element, first sterile filter
22 second sterile filter element, second sterile filter
22a sterile filter element
22b sterile filter element
30 first fluid reservoir
31 first fluid
40 second fluid reservoir
41 second fluid
50 container, bag
51 mixture of a first fluid and a second fluid
60 sterile enclosure, sterile bag sterile environment
71 connector element, connector
72 connector element, connector
73 connector element, connector
74 connector element, connector
75 connector element, connector
76 connector element, connector
77 connector element, connector
78 connector element, connector
79 connector element, connector
81 valve element, stopcock, 3-way-valve
82 valve element, stopcock, 3-way-valve
83 valve element, clamp
84 valve element, clamp
84a valve element, clamp
84b valve element, clamp
85 valve element, clamp
86 valve element, clamp
86a valve element, clamp
86b valve element, clamp
87 valve element, clamp
100 fluid path system
200 pumping unit, syringe pump
201 first syringe element, first syringe
202 second syringe element, second syringe
202a syringe element
202b syringe element
300 rocker device
400 pump
500 pump
600 reservoir for a further substance, for example a cryoprotectant
700 filter element
800 container, bag
900 method
901-914 method steps
1000 mixing system
A arrow
B arrow
Ba arrow
Bb arrow
C arrow
D arrow
Da arrow
Db arrow

1. A fluid path system, in particular for aseptic processing of at least a first fluid and a second fluid, the fluid path system comprising:
at least a first tubing unit and a second tubing unit,
wherein the first tubing unit is configured to receive the first fluid and the second tubing unit is configured to receive the second fluid,
a mixing element configured to combine the first fluid and the second fluid to provide a mixture of the first fluid and the second fluid and to discharge the mixture of the first fluid and the second fluid, and
at least one sterile filter unit configured to allow passage of an auxiliary medium, such that a sterile auxiliary medium is supplied or can be supplied to the first tubing unit and to the second tubing unit.

2. A fluid path system, in particular for aseptic processing of at least a first fluid and a second fluid, the fluid path system comprising:
at least a first tubing unit and a second tubing unit,
wherein the first tubing unit is configured to receive the first fluid and the second tubing unit is configured to receive the second fluid,
a mixing element configured to combine the first fluid and the second fluid to provide a mixture of the first fluid and the second fluid and to discharge the mixture of the first fluid and the second fluid, and
at least one sterile filter unit configured to allow passage of a sterile auxiliary medium for transmitting or applying a pressure to the tubing units, such that the first fluid and the second fluid are pumpable through the fluid path system and that the mixture of the first fluid and second fluid is provided.

3. A fluid path system, in particular for aseptic processing of at least a first fluid and a second fluid, the fluid path system comprising:
at least a first tubing unit and a second tubing unit,
wherein the first tubing unit is configured to receive the first fluid and the second tubing unit is configured to receive the second fluid,
a mixing element configured to combine the first fluid and the second fluid to provide a mixture of the first fluid and the second fluid and to discharge the mixture of the first fluid and the second fluid, and
at least one sterile filter unit configured to allow passage of a sterile auxiliary medium for transmitting or applying a pressure to the tubing units, such that the first fluid is receivable in the first tubing unit, the second fluid is receivable in the second tubing unit, the first fluid and the second fluid are combinable in the mixing to element and/or the mixture is dischargeable from the mixing element.

4. A fluid path system, in particular for aseptic processing of at least a first fluid and a second fluid, the fluid path system comprising:
at least a first tubing unit and a second tubing unit,
wherein the first tubing unit is configured to receive the first fluid and the second tubing unit is configured to receive the second fluid,
a mixing element configured to combine the first fluid and the second fluid to provide a mixture of the first fluid and the second fluid and to discharge the mixture of the first fluid and the second fluid, and
at least one sterile filter unit configured to allow passage of a sterile auxiliary medium which mediates one or more of the following: filling the first tubing unit with the first fluid, filling the second tubing unit with the second fluid, combining the first fluid and the second fluid in the mixing element, and discharging the mixture of the first fluid and the second fluid from the mixing element.

5. The fluid path system according to any one of claims 1 to 4, wherein one of the fluids is a liposome colloid and the other fluid is an RNA solution, optionally a NaCl containing RNA solution.

6. The fluid path system according to any one of claims 1 to 5, wherein the auxiliary medium comprises or is air or comprises or is a gas.

7. The fluid path system according to any one of claims 1 to 6, wherein at least the first tubing unit, the second tubing unit and the mixing element are provided as a tubing set.

8. The fluid path system according to any one of claims 1 to 7, wherein the fluid path system and/or the tubing set is/are pre-assembled.

9. The fluid path system according to any one of claims 1 to 8, wherein the fluid path system is a sterile fluid path system or is a pre-sterilized fluid path system.

10. The fluid path system according to any one of claims 1 to 9, wherein the at least one sterile filter unit is configured to supply a sterile auxiliary medium to the first tubing unit and to the second tubing unit.

11. The fluid path system according to any one of claims 1 to 10, wherein the at least one sterile filter unit is configured to prevent passage of the first fluid and/or the second fluid.

12. The fluid path system according to any one of claims 1 to 11, wherein the at least one sterile filter unit is configured to allow the passage of the auxiliary medium for transmitting a pressure to the tubing units, in particular to the first fluid and second fluid, such that the first fluid is receivable in the first tubing unit, the second fluid is receivable in the second tubing unit, the first fluid and the second fluid are combinable in the mixing element and/or the mixture is dischargeable from the mixing element.

13. The fluid path system according to any one of claims 1 to 12, wherein the at least one sterile filter unit comprises one or more sterile filter elements, in particular at least a first sterile filter element and a second sterile filter element,
wherein, optionally, the at least one sterile filter unit comprises
i) a first sterile filter element associated with the first tubing unit and a second sterile filter element associated with the second tubing unit, or
ii) a sterile filter element associated with the first tubing unit and the second tubing unit.

14. The fluid path system according to any one of claims 1 to 13, wherein the fluid path system is connectable to a pumping unit, so that the fluids to be mixed can be pumped through the fluid path system.

15. The fluid path system according to any one of claims 1 to 14, wherein the fluid path system is connectable to a or the pumping unit, so that the auxiliary medium is suppliable or can be supplied to or is removable or can be removed from the tubing units.

16. The fluid path system according to any one of claims 1 to 15, wherein the fluid path system, in particular the first tubing unit and the second tubing unit, are connectable to a or the pumping unit, in particular via the sterile filter unit, for supplying the auxiliary medium to or removing the auxiliary medium from the tubing units.

17. The fluid path system according to any one of claims 1 to 16, in particular according to any one of claims 14 to 16, wherein the pumping unit comprises two or more syringe elements, in particular at least a first syringe element and a second syringe element,
wherein, optionally, the first tubing unit is connectable to the first sterile filter element and the first sterile filter element is connectable to the first syringe element, and
the second tubing unit is connectable to the second sterile filter element and the second sterile filter element is connectable to the second syringe element.

18. The fluid path system according to any one of claims 1 to 17, in particular according to claim 17, wherein the pumping unit is configured to drive the syringe elements in parallel.

19. The fluid path system according to any one of claims 1 to 18, further comprising two or more fluid reservoirs, in particular a first fluid reservoir and a second fluid reservoir,
wherein, optionally, the first fluid reservoir containing the first fluid is connectable to the first tubing unit and the second fluid reservoir containing the second fluid is connectable to the second tubing unit.

20. The fluid path system according to any one of claims 1 to 19, further comprising at least one container or product bag for receiving the mixture of the first fluid and the second fluid, wherein the at least one container is connectable to the mixing element.

21. The fluid path system according to any one of claims 1 to 20, further comprising one or more sterile connector elements,
wherein, optionally, the connector elements are configured for connecting the first fluid reservoir for the first fluid to the first tubing unit, the second fluid reservoir for the second fluid to the second tubing unit, the at least one container for the mixture of the first fluid and the second fluid to the mixing element, and/or the sterile filter unit to the pumping unit.

22. The fluid path system according to any one of claims 1 to 21, further comprising one or more connecting tubes which are configured to connect the components or elements of the fluid path system to each other or to external components or elements.

23. The fluid path system according to any one of claims 1 to 22, further comprising one or more valve elements, wherein at least one valve element is arranged
i) between each tubing unit and an associated fluid reservoir, and/or
ii) between each tubing unit and the mixing element, and/or
iii) between the mixing element and the container for the mixture of the first fluid and the second fluid.

24. The fluid path system according to any one of claims 1 to 23, further comprising one or more valve elements for allowing and/or preventing one or more of the following: the input of the first fluid, the input of the second fluid, the combination of the first fluid and the second fluid, and the output of the mixture of the first fluid and the second fluid.

25. The fluid path system according to any one of claims 1 to 24, further comprising one or more valve elements for allowing and/or preventing one or more of the following: filling the first tubing unit with the first fluid, filling the second tubing unit with the second fluid, combining the first fluid and the second fluid in the mixing element, and discharging the mixture of the first fluid and the second fluid from the mixing element.

26. The fluid path system according to any one of claims 1 to 25, in particular according to any one of claims 23 to 25, wherein the one or more valve elements comprise one or more of the following: a three-way valve, a T-piece, a clamp, and a pinch valve.

27. The fluid path system according to any one of claims 1 to 26, wherein the mixing element comprises a manifold valve, a mixing tee, a Y-mixer or a T-mixer.

28. The fluid path system according to any one of claims 1 to 27, wherein one or more of the following are single use elements: the at least one first tubing unit or at least a part of the first tubing unit, the at least one second tubing unit or at least a part of the second tubing unit, the mixing element, the at least one sterile filter unit, the one or more valve elements and the one or more connector elements.

29. The fluid path system according to any one of claims 1 to 28, wherein one or more of the following are sterile elements: the at least one first tubing unit, the at least one second tubing unit, the mixing element, the at least one sterile filter unit, the one or more valve elements and the one or more connector elements.

30. The fluid path system according to any one of claims 1 to 29, wherein the first tubing unit is configured for complete input of the first fluid and/or the second tubing unit is configured for complete input of the second fluid.

31. The fluid path system according to any one of claims 1 to 30, wherein the first tubing unit and/or the second tubing unit comprise(s) a storage capacity such that a predetermined amount of a respective fluid is storable within the first tubing unit and/or the second tubing unit.

32. The fluid path system according to any one of claims 1 to 31, wherein each tubing unit comprises one or more tube elements or tubes.

33. The fluid path system according to any one of claims 1 to 32, wherein each tubing unit comprises at least two tube elements which are both connectable to a respective fluid reservoir or to different fluid reservoirs, so that a continuous flow of fluid can be provided.

34. The fluid path system according to any one of claims 1 to 33, further comprising a sterile enclosure or sterile bag, wherein at least the first tubing unit, the second tubing unit and/or the mixing element are arranged within the sterile enclosure or sterile bag.

35. The fluid path system according to any one of claims 1 to 34, in particular according to any one of claims 19 to 34, wherein the two or more fluid reservoirs and/or the at least one container is/are arranged within the sterile enclosure or sterile bag.

36. The fluid path system according to any one of claims 1 to 35, wherein the fluid path system comprises pharma grade soft tubes and/or pharma grade bags.

37. The fluid path system according to any one of claims 1 to 36, wherein the ratio of the volumes of the at least two fluids is 1 to 1.

38. The fluid path system according to any one of claims 1 to 37, wherein the at least first tubing unit and second tubing unit comprise the same shape, size, volume and/or storage capacity.

39. A mixing system comprising:
the fluid path system according to any one of claims 1 to 38, and
a pumping unit for pumping a first fluid and a second fluid through the fluid path system.

40. The mixing system according to claim 39, wherein the pumping unit is configured to provide a pressure or a pressure gradient, in particular a pneumatic pressure gradient, in particular via the auxiliary medium, to the fluid path system, in particular to the tubing units, in particular to the fluids to be mixed.

41. The mixing system according to claim 39 or claim 40, wherein the pumping unit comprises two or more syringe elements, in particular at least a first syringe element and a second syringe element,
wherein, optionally, the first tubing unit is connectable to the first sterile filter element and the first sterile filter element is connectable to the first syringe element, and
the second tubing unit is connectable to the second sterile filter element and the second sterile filter element is connectable to the second syringe element.

42. The mixing system according to any one of claims 39 to 41, wherein each tubing unit comprises at least two tube elements which are both connectable to a respective fluid reservoir or to different fluid reservoirs, and wherein each tube element is connectable to a syringe element, wherein each tube element is connectable to a different syringe element, so that preferably a continuous flow of fluid can be provided.

43. The mixing system according to any one of claims 39 to 42, further comprising
(i) a second pumping unit, optionally a peristaltic pump, for supplying a substance to the at least one container, and/or
(ii) a rocker device, wherein the at least one container is receivable in the rocker device, and/or
(iii) a third pumping unit, optionally a peristaltic pump, for pumping the mixture from the container to a further container, optionally via a further filter element.

44. The mixing system according to any one of claims 39 to 43, in particular according to any one of claims 41 to 43, wherein the pumping unit is configured to drive the syringe elements in parallel.

45. The mixing system according to any one of claims 39 to 44, wherein the mixing system is configured to manufacture the mixture with one pumping cycle.

46. The mixing system according to any one of claims 39 to 45, further comprising instructions for use of the fluid path system and/or the mixing system.

47. The mixing system according to any one of claims 39 to 46, wherein one of the fluids is a liposome colloid and the other fluid is an RNA solution, optionally a NaCl containing RNA solution.

48. The mixing system according to any one of claims 39 to 47, wherein the at least first tubing unit and second tubing unit comprise the same shape, size, volume and/or storage capacity.

49. Use of the fluid path system according to any one of claims 1 to 38 for aseptic manufacturing of a sterile mixture of at least a first fluid and a second fluid.

50. Use of at least one sterile filter unit as part of the fluid path system according to any one of claims 1 to 38 for aseptic manufacturing of a sterile mixture of at least a first fluid and a second fluid.

51. Use of the mixing system according to any one of claims 39 to 48 for aseptic manufacturing of a sterile mixture of at least a first fluid and a second fluid.

52. A method for aseptic manufacturing of a sterile mixture of at least a first fluid and a second fluid, the method comprising the steps of:
filling at least a first tubing unit with the first fluid and a second tubing unit with the second fluid by removing an auxiliary medium, in particular in parallel, from the tubing units,
combining the first fluid and the second fluid in a mixing element and discharging a mixture of the first fluid and the second fluid from the mixing element by supplying the auxiliary medium, in particular in parallel, via at least one sterile filter unit, preferably via a first sterile filter element associated with the first tubing unit and a second sterile filter element associated with the second tubing unit, to the tubing units.

53. A method for aseptic manufacturing of a sterile mixture of at least a first fluid and a second fluid by using the fluid path system according to any one of claims 1 to 38 or the mixing system according to any one of claims 39 to 48, the method comprising the steps of:
filling the at least first tubing unit with the first fluid and the second tubing unit with the second fluid by removing the auxiliary medium, in particular in parallel, from the tubing units,
combining the first fluid and the second fluid in the mixing element and discharging a mixture of the first fluid and the second fluid from the mixing element by supplying the auxiliary medium, in particular in parallel, via the at least one sterile filter unit, preferably via the first sterile filter element associated with the first tubing unit and the second sterile filter element associated with the second tubing unit, to the tubing units.

54. The method according to claim 52 or claim 53, further comprising the step of connecting the first tubing unit and the second tubing unit to the or a pumping unit for pumping the first fluid and the second fluid.

55. The method according to any one of claims 52 to 54, further comprising the steps of
opening the fluid path between the or a first fluid reservoir and the first tubing unit and opening the fluid path between the or a second fluid reservoir and the second tubing unit, and
closing the fluid path between the first tubing unit and the mixing element and closing the fluid path between the second tubing unit and the mixing element
for filling the first tubing unit with the first fluid and filling the second tubing unit with the second fluid.

56. The method according to any one of claims 52 to 55, further comprising the steps of
closing the fluid path between the or a first fluid reservoir and the first tubing unit and closing the fluid path between the or a second fluid reservoir and the second tubing unit, and
opening the fluid path between the first tubing unit and the mixing element and opening the fluid path between the second tubing unit and the mixing element
for combining the first fluid and the second fluid and for discharging the mixture of the first fluid and the second fluid from the mixing element.
